(19) United States
(12) Patent Application Publication
(10) Pub. No.: US 2025/0058017 A1
(43) Pub. Date: Feb. 20, 2025

(54) EXTRACELLULAR MATRIX-BASED HYBRID INK FOR 3D PRINTING AND METHOD FOR MANUFACTURING THE SAME

(71) Applicant: POSTECH RESEARCH AND BUSINESS DEVELOPMENT FOUNDATION, Pohang-si Gyeongsangbuk-do (KP)

(72) Inventors: Jin Ah JANG, Pohang-si (KP); Hyeon Ji KIM, Changwon-si (KP); Seung Hyeon HWANG, Pohang-si (KP)

(73) Assignee: POSTECH RESEARCH AND BUSINESS DEVELOPMENT FOUNDATION, Pohang-si Gyeongsangbuk-do (KP)

(21) Appl. No.: 18/721,363

(22) PCT Filed: Mar. 14, 2022
(86) PCT No.: PCT/KR2022/003518

§ 371 Date: Jun. 18, 2024

(30) Foreign Application Priority Data
Dec. 20, 2021 (KR) 10-2021-0182375

(51) Int. Cl. A61L 27/20; A61L 27/22; A61L 27/36; A61L 27/56

(52) U.S. Cl. CPC A61L 27/20; A61L 27/227; A61L 27/3633; A61L 27/3687; A61L 27/56

(57) ABSTRACT

One embodiment of the present disclosure provides an extracellular matrix-based hybrid ink for 3D printing, the hybrid ink including an extracellular matrix having an amine group and a modified hyaluronic acid introduced with an ethylenically unsaturated bond functional group, in which the extracellular matrix and the modified hyaluronic acid are in a crosslinked state by a chemical bond formed between at least some of amine groups present in the extracellular matrix and the ethylenically unsaturated bond functional group present in the modified hyaluronic acid. According to an embodiment of the present disclosure, the hybrid ink has mechanical properties suitable for 3D printing through chemical crosslinking between the extracellular matrix and modified hyaluronic acid as constituents, and can control the drug release rate by adjusting the chemical crosslinking density.

### TECHNICAL FIELD

The present disclosure relates to an extracellular matrix-based hybrid ink and the like, and more particularly, to an extracellular matrix-based hybrid ink for 3D printing and a method for manufacturing the same, having mechanical properties suitable for 3D printing and capable of controlling a drug release rate by controlling the crosslinking density through chemical bonding between internal components.

### BACKGROUND ART

Cerebral ischemia occurs when insufficient blood flow to the brain leads to cerebral hypoxia and death of brain tissue. Because of the severity, the cerebral ischemia is known as one of the most common causes of death worldwide. For the treatment of ischemic brain tissue, it is essentially required to maintain the patency of newly formed vessels and induce angiogenesis, which generates new vessels by newly extending capillaries from existing vessels, through continuous supply of angiogenic growth factors. The angiogenesis process is a complex multi-stage process including a variety of angiogenic growth factors that are essential at the right time and serves to continuously supply oxygen and nutrients necessary for cell invasion and metabolic maintenance. A treatment method of delivering angiogenic factors to promote angiogenesis is emerging as promising strategies for the treatment of severe ischemic

Angiogenic factors that promote the early and mature stages of therapeutic angiogenesis are well known. In addition, administration of angiogenic factors acting in each stage is also recognized as a beneficial method for promoting revascularization. In particular, a vascular endothelial growth factor (VEGF) is one of the important factors that promote the early stages of vascularization, and induces generation of immature vessels by proliferating endothelial cells. However, concerns about a clinical use of VEGF have been raised due to proinflammatory responses, such as increased vascular permeability, as side effects of VEGF. A hepatocyte growth factor (HGF) is another important angiogenic factor that promotes the maturation stage of angiogenesis, and causes a synergistic effect when applied together with VEGF, such as reducing the side effects of VEGF and promoting the growth of vascular endothelial cells. Accordingly, a sequential release process of the two angiogenic factors induces a more robust angiogenic response, and prevents vascular regression or blood leakage. Therefore, continuous administration of these multiple angiogenic factors that mimic the angiogenic process is essential for angiogenesis in ischemic areas.

However, traditional growth factor administration methods, such as intravenous injection, often require high doses or repeated delivery to stimulate therapeutic effects, resulting in reduced efficacy and serious side effects. To solve the problem, these factors need to be administered at a physiologically appropriate time (time control) in an appropriate site (site-specific) targeted manner. Therefore, there is a need to develop a soft and flexible hydrogel patch-type drug delivery system that may control a release pattern of the angiogenic factors and be applied locally to vulnerable brain regions. 3D printing technology may be used to manufacture these patches. By using 3D printing technology and various types of biomaterials, controllable and customizable patch-type delivery systems may be freely manufactured. The control of the release pattern of the angiogenic factors may be achieved by adjusting 3D printing parameters (e.g., dimensions and design of system) or controlling material properties (e.g., synthetic and natural biomaterials, crosslinking density and concentration). For biomaterials, extracellular matrix (ECM)-based hydrogels with tissue properties and printability have been proposed as a practicable option.

According to recent studies, the usability of vessel-derived decellularized extracellular matrix (VdECM) as a drug delivery material has been reported, but there is a disadvantage in that it is difficult to use VdECM alone as a printable biomaterial ink due to low printability (G. Gao, J. Y. Park, B. S. Kim, J. Jang, D. W. Cho, Advanced healthcare materials 2018, 7, 1801102.).

Regarding an extracellular matrix-based bioink for 3D printing, in Korean Patent Publication No. 10-1954953, there is disclosed a method for manufacturing an artificial tissue: including steps of (a) preparing a thermosensitive hydrogel containing a cell culture medium, hyaluronic acid, gelatin, fibrinogen and glycerol: (b) preparing a decellularized extracellular matrix (dECM) powder by decellularizing a tissue extracted from the body and pulverizing the tissue into a particle size of 0.05 to 100 μm: (c) solating the thermosensitive hydrogel obtained in step (a) at 30 to 40° C.: (d) preparing a decellularized extracellular matrix-based bioink by mixing the dECM powder obtained in step (b) with the sol-state thermosensitive hydrogel of step (c) through stirring: (e) mixing the dECM-based bioink prepared in step (d) and cells derived from a desired tissue and then injecting the mixture into a cartridge for a 3D bioprinter: (f) maintaining the cartridge for the 3D bioprinter of step (e) at 3 to 6° C. for 8 to 15 minutes to induce gelation according to temperatures of the cells and the dECM-based bioink within the cartridge: (g) 3D bioprinting a desired artificial tissue using the cartridge for the 3D bioprinter containing the gel-induced cells and dECM-based bioink of step (f); and (h) treating a thrombin solution to the 3D bioprinted desired artificial tissue in step (g) and inducing gelation through enzymatic polymerization of thrombin and fibrinogen by maintaining at 20 to 25° C. for 25 to 35 minutes. Further, in Korean Patent Registration No. 10-2302770, there is disclosed a bioink composition for 3D printing including a microparticlized human tissue-derived component and a biocompatible polymer, in which the particle size of the microparticlized human tissue-derived component is 10 to 1000 μm.

### DISCLOSURE

### Technical Problem

The present disclosure has been derived from the technical background of the related art, and an object of the present disclosure is to provide an extracellular matrix-based hybrid ink for 3D printing and a method for manufacturing the same, having mechanical properties suitable for 3D printing and capable of controlling a drug release rate.

Another object of the present disclosure is to provide a biomaterial-based drug delivery patch and a method for manufacturing the same.

Yet another object of the present disclosure is to provide a biomaterial-based patch that can be used to promote angiogenesis or treat angiogenesis-related diseases.

### Technical Solution

The present inventors developed an extracellular matrix-based hybrid ink having mechanical properties suitable for 3D printing and capable of controlling a drug release rate by chemically bonding an amine group of an extracellular matrix with a methacrylate group of a methacrylated hyaluronic acid through an aza-Michael addition reaction. In addition, the present inventors manufactured a hydrogel patch by adding angiogenic growth factors capable of promoting cerebral angiogenesis to the extracellular matrix-based hybrid ink and then 3D printing the mixture so that the angiogenic growth factors are spatially compartmentalized and sequentially released. The present inventors confirmed that a patch in which angiogenic growth factors are spatiotemporally compartmentalized was transplanted into the brain and then monitored using a photoacoustic microscope and as a result, the patch effectively promoted the angiogenesis.

In order to achieve the object, one embodiment of the present disclosure provides an extracellular matrix-based hybrid ink for 3D printing as the hybrid ink including an extracellular matrix having an amine group and a modified hyaluronic acid introduced with an ethylenically unsaturated bond functional group, in which the extracellular matrix and the modified hyaluronic acid are in a crosslinked state by a chemical bond formed between at least some of amine groups present in the extracellular matrix and the ethylenically unsaturated bond functional group present in the modified hyaluronic acid.

In order to achieve the object, one embodiment of the present disclosure provides a method for manufacturing an extracellular matrix-based hybrid ink for 3D printing including: preparing an extracellular matrix solution having a pH of 6.5 to 7.5; and adding and uniformly mixing a modified hyaluronic acid introduced with an ethylenically unsaturated bond functional group to the extracellular matrix solution, and then performing an aza-Michael addition reaction to obtain an ink composition in which the extracellular matrix and the modified hyaluronic acid are in a crosslinked state.

In order to achieve the object, one embodiment of the present disclosure provides a biomaterial-based drug delivery patch as a multilayer patch including an inner core layer and an outer layer surrounding the inner core layer, in which the inner core layer and the outer layer are made of extracellular matrix-based hybrid inks, the hybrid ink includes an extracellular matrix having an amine group, a modified biopolymer introduced with an ethylenically unsaturated bond functional group, and a growth factor, the extracellular matrix and the modified biopolymer constituting the hybrid ink are present in a chemically crosslinked state, and the extracellular matrix-based hybrid ink constituting the inner core layer and the extracellular matrix-based hybrid ink constituting the outer layer have different chemical crosslinking densities and include different growth factors.

In order to achieve the object, one embodiment of the present disclosure provides a method for manufacturing a biomaterial-based drug delivery patch including: preparing a first hybrid ink consisting of a mixture including an extracellular matrix having an amine group, a modified biopolymer introduced with an ethylenically unsaturated bond functional group, and a first growth factor, in which the extracellular matrix and the modified biopolymer are present in a chemically crosslinked state: preparing a second hybrid ink consisting of a mixture including an extracellular matrix having an amine group, a modified biopolymer introduced with an ethylenically unsaturated bond functional group, and a second growth factor different from the first growth factor, in which the extracellular matrix and the modified biopolymer are present in a chemically crosslinked state, and having a different chemical crosslinking density from the first hybrid ink: forming an inner core layer by 3D printing the second hybrid ink; and forming an outer layer surrounding the inner core layer by 3D printing the first hybrid ink and obtaining a multilayer structure.

In order to achieve the object, one embodiment of the present disclosure provides a biomaterial-based angiogenesis promoting patch as a multilayer patch including an inner core layer and an outer layer surrounding the inner core layer, in which the outer layer is made of an extracellular matrix-based first hybrid ink, and the inner core layer is made of an extracellular matrix-based second hybrid ink, the first hybrid ink includes an extracellular matrix having an amine group, a modified biopolymer introduced with an ethylenically unsaturated bond functional group, and a first growth factor that promotes an early stage of angiogenesis, and the second hybrid ink includes an extracellular matrix having an amine group, a modified biopolymer introduced with an ethylenically unsaturated bond functional group, and a second growth factor that promotes a mature stage of angiogenesis, the extracellular matrix and the modified biopolymer constituting the first hybrid ink and the second hybrid ink are present in a chemically crosslinked state, and the second hybrid ink has a higher chemical crosslinking density than the first hybrid ink.

In order to achieve the object, one embodiment of the present disclosure provides a method for manufacturing a biomaterial-based angiogenesis promoting patch including: preparing a first hybrid ink consisting of a mixture including an extracellular matrix having an amine group, a modified biopolymer introduced with an ethylenically unsaturated bond functional group, and a first growth factor that promote an early stage of angiogenesis, in which the extracellular matrix and the modified biopolymer are present in a chemically crosslinked state: preparing a second hybrid ink consisting of a mixture including an extracellular matrix having an amine group, a modified biopolymer introduced with an ethylenically unsaturated bond functional group, and a second growth factor that promotes a mature stage of angiogenesis, in which the extracellular matrix and the modified biopolymer are present in a chemically crosslinked state, and having a higher chemical crosslinking density than the first hybrid ink; forming an inner core layer by 3D printing the second hybrid ink; and forming an outer layer surrounding the inner core layer by 3D printing the first hybrid ink and obtaining a multilayer structure.

The biomaterial-based angiogenesis promoting patch may be used to treat angiogenesis-related diseases.

### Advantageous Effects

According to an embodiment of the present disclosure, the hybrid ink has mechanical properties suitable for 3D printing through chemical crosslinking between the extracellular matrix and the modified hyaluronic acid as constituents, and can control the drug release rate by adjusting the chemical crosslinking density. According to an embodiment of the present disclosure, the multilayer patch in which different growth factors are spatiotemporally compartmentalized, manufactured by 3D printing the hybrid ink, sequentially sustained-releases different growth factors, and thus can be used for a drug delivery system, promoting angiogenesis or treating angiogenesis-related diseases.

### DESCRIPTION OF DRAWINGS

FIG. 1 illustrates a schematic process of manufacturing a hybrid ink (HAVEM ink) from vessel-derived decellularized extracellular matrix (VdECM) and methacrylated hyaluronic acid (HAMA) using a dual crosslinking mechanism such as chemical crosslinking and thermal crosslinking, in the present study.

FIG. 2 illustrates a schematic process of manufacturing a spatiotemporal compartmentalized cerebral angiogenesis inducing (SCAI) patch using a 3D printing process by adding VEGF and HGF, which are cerebral angiogenesis inducing growth factors, to hybrid inks with different crosslinking densities, respectively, and a schematic process of sequentially releasing the VEGF and HGF from the SCAI patch, in the present study.

FIG. 3 schematically illustrates a process of manufacturing an HAVEM ink from vessel-derived decellularized extracellular matrix (VdECM) and methacrylated hyaluronic acid (HAMA) through an aza-Michael addition reaction.

FIG. 4 illustrates spectra measured by nuclear magnetic resonance (NMR) with respect to a VdECM ink (4V0H), HAMA, and an HAVEM ink (4V0.5H) to demonstrate the occurrence of crosslinking between vessel-derived decellularized extracellular matrix (VdECM) and methacrylated hyaluronic acid (HAMA).

An upper photograph of FIG. 5 shows sol-state images of VdECM ink (4V0H), HAVEM ink (4V0.5H), and HAVEM ink (4V1H) according to a difference in chemical crosslinking density, and a lower graph of FIG. 5 shows a result of measuring viscosities of VdECM ink (4V0H), HAVEM ink (4V0.5H), and HAVEM ink (4V1H) under printing conditions of 4° C. and various shear rates.

FIG. 6 illustrates changes in elastic modulus of VdECM ink (4V0H), HAVEM ink (4V0.5H), and HAVEM ink (4V1H) according to a temperature change from 4° C. to 37° C.

FIG. 7 illustrates storage modulus and loss modulus of VdECM ink (4V0H), HAVEM ink (4V0.5H), and HAVEM ink (4V1H) according to a change in frequency at a temperature of 37° C.

FIG. 8 shows a result of measuring release profiles of HGF encapsulated in HAVEM ink (4V0.5H) and HAVEM ink (4V1H) for 7 days.

FIG. 9 schematically illustrates a process of manufacturing an SCAI patch using 3D printing technology in the present study.

FIG. 10 shows a result of measuring an angiogenesis effect of various angiogenic factors by a tube formation assay.

FIG. 11 shows images of a cross-sectional structure and each cross-section of the SCAI patch manufactured in the present study photographed using a confocal microscope (scale bars: 200 μm).

FIG. 12 shows images of design structures of SCAI patches manufactured with various combinations of HAVEM inks and images of respective patches in printing, gelation, and vitrification steps.

FIG. 13 shows tensile strength measurement results and swelling ratios of the SCAI patch through the vitrification process manufactured in the present study.

FIG. 14 shows SEM photographed images of various vitrified patches manufactured in the present study (scale bars: 1 μm). In FIG. 14, “4VOH” represents a vitrified patch made of VdECM ink (4VOH), “4V0.5H” represents a vitrified patch made of HAVEM ink (4V0.5H), and “4V1H” represents a vitrified patch made of HAVEM ink (4V1H).

FIG. 15 schematically shows a structure of a multilayer SCAI patch designed in the present study for sequential drug sustained-release of VEGF and HGF.

FIG. 16 shows a result of evaluating cumulative release profiles of VEGF and HGF for one month for two multilayer SCAI patches designed in the present study for sequential drug sustained-release of VEGF and HGF, respectively.

FIG. 17 shows an in vitro experimental result when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs).

FIG. 18 shows a result of analyzing cell viability using a live/dead assay kit when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs) (scale bars: 50 μm).

FIG. 19 shows a result of analyzing expression levels of genes related to angiogenesis when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs).

FIG. 20 schematically illustrates an in vivo experiment consisting of a process of transplanting an SCAI patch spatiotemporally compartmentalized with VEGF and HGF manufactured in the present study into the cerebral cortex of SD rats and then monitoring a transplanted area using an OR-PAM system.

FIG. 21 shows images of cross-sections of an area where a patch is transplanted photographed using a photoacoustic (PA) B-mode in order to track changes in the SCAI patch over time in an in vivo experiment for evaluating a cerebral angiogenesis behavior after SCAI patch transplantation conducted in the present study (scale bars: 200 μm).

FIG. 22 shows changes in brain vascular structure in SD rats using photoacoustic (PA) maximum amplitude projection (MAP) images according to whether or not to be treated with SCAI patches in an in vivo experiment to evaluate cerebral angiogenesis behavior after SCAI patch transplantation performed in the present study.

FIG. 23 is a cross-sectional PA B-mode image for areas C1 and C2 indicated by dotted lines in the image of a rat applied with the SCAI patch in FIG. 22 (scale bars: 400 μm). In the enlarged image of FIG. 23 (scale bars: 1 mm), large vessels are highlighted by elliptical curves formed by dots below.

FIG. 24 shows results of quantitative analysis of the vessel density of SD rats according to whether or not to be treated with SCAI patches using optical resolution photoacoustic microscopy (OR-PAM) images in an in vivo experiment to evaluate a cerebral angiogenesis behavior after SCAI patch transplantation performed in the present study.

FIG. 25 shows histological analysis results for a patch-untreated group and an SCAI patch-transplanted group after 14 days of SCAI patch transplantation in H&E staining images and immunohistochemical (IHC) images (CD 31) in an in vivo experiment to evaluate a cerebral angiogenesis behavior after SCAI patch transplantation performed in the present study (scale bars: 50 μm).

FIG. 26 shows results of quantitative analysis of the number of vessels per unit area in a patch-untreated group and an SCAI patch-transplanted group using immunohistochemical (IHC) images after 14 days of SCAI patch transplantation in an in vivo experiment to evaluate a cerebral angiogenesis behavior after SCAI patch transplantation performed in the present study.

### MODES OF THE INVENTION

Hereinafter, the present disclosure will be described in detail.

As used herein, the term ‘bioink’ is defined as a cell compatible material capable of 3D printing. The bioink may be extruded through a needle at 0 to 37° C. and then gelled or solidified. The bioink may be formulated to be suitable for inkjet, laser-assisted, or microvalve 3D printing devices.

As used herein, the term ‘extracellular matrix (ECM)’ is an extracellular portion of animal tissue that performs various other important functions while generally providing a structural support to animal cells. The ECM is a characteristic that defines a connective tissue in an animal and consists of various types of proteins, including collagen, glycosaminoglycan (GAG), etc. The ECM may be a tissue of animals such as pigs and cows and can be extracted from various organs.

As used herein, the term ‘biopolymer’ is a general term for polymer compounds synthesized and produced within the body of a living organism, and representative examples thereof include proteins, nucleic acids, polysaccharides, etc., and specific materials include hyaluronic acid, collagen, etc.

As used herein, the term ‘crosslinking density’ is defined as a ratio of the number of crosslinking structural units (crosslinking points) to the total number of structural units in a crosslinking polymer. The physical properties of crosslinking polymer gels, such as swelling and elasticity, change significantly depending on the crosslinking density.

As used herein, the term ‘growth factor’ is a protein involved in regulating the growth and differentiation of cells or organisms, and acts as a signaling substance that transmits signals into the cell by binding to a specific receptor present on the cell surface, and regulates various cellular physiological phenomena such as differentiation, regeneration, and healing of the cell. Specific examples of the growth factor include vascular endothelial growth factor (VEGF), hepatocyte growth factor (HGF), epithermal growth factor (EGF), insulin-like growth factor (IGF), erythropoietin (EPO), fibroblast growth factor (FGF), brain-derived neurotrophic factor (BDNF), nerve growth factor (NGF), keratinocyte growth factor (KGF), interleukin (IL), colony-stimulating factor (CSF), angiopoietin (ANG), platelet-derived growth factor (PDGF), placental growth factor (PGF), transforming growth factor-α (TGF-α), transforming growth factor-β (TGF-β), matrix metalloproteinase (MMP), bone morphogenetic protein (BMP), and the like.

One aspect of the present disclosure relates to an extracellular matrix-based hybrid ink for 3D printing and a method for manufacturing the same, which has mechanical properties suitable for 3D printing and is able to control a drug release rate.

The hybrid ink according to an embodiment of the present disclosure may be classified as a bioink or a biomaterial ink since it is based on a biomaterial that is a cell compatible material. In addition, the hybrid ink according to an embodiment of the present disclosure is in the form of a composition in which the components are uniformly mixed, and exists in a sol state or an intermediate state between a sol and a hydrogel in order to have appropriate fluidity required for 3D printing.

The extracellular matrix-based hybrid ink for 3D printing according to an embodiment of the present disclosure includes an extracellular matrix having an amine group and a modified hyaluronic acid introduced with an ethylenically unsaturated bond functional group.

The extracellular matrix may be preferably derived from various organ tissues depending on the purpose of use of the hybrid ink, and is preferably derived from vascular tissue when considering the purpose of manufacturing a patch for promoting angiogenesis, etc. to be described below. In addition, when considering the use for biotransplantation, etc., the extracellular matrix is preferably a decellularized extracellular matrix. It is effective in minimizing the immune response in allograft or xenograft by removing cells capable of acting as an antigen that induces the immune response through the decellularization process. Since the type and number of cells and the physical properties of the tissue itself vary depending on a tissue, decellularization is performed using various chemicals such as acids, bases, storage solutions, hypertonic solutions, detergents, etc. In addition, the extracellular matrix may be used while maintaining the structure of the tissue itself only through the decellularization process, but may be used by being dissolved in an acidic solution through the freeze-drying and pulverization processes, or being into a sol-state solution through the neutralization process again.

The ethylenically unsaturated bond functional group introduced into the modified hyaluronic acid is not particularly limited in the type as long as it may undergo an aza-Michael addition reaction with an amine group present in the extracellular matrix, and may be selected from, for example, a vinyl group, an allyl group, an acrylic group, a methacrylic group, etc. In the extracellular matrix-based hybrid ink for 3D printing according to an embodiment of the present disclosure, the modified hyaluronic acid may be selected from methacrylated hyaluronic acid or acrylated hyaluronic acid, considering biocompatibility, reactivity with amine groups, etc. The methacrylated hyaluronic acid has a chemical structure represented by the following Chemical Formula 1.

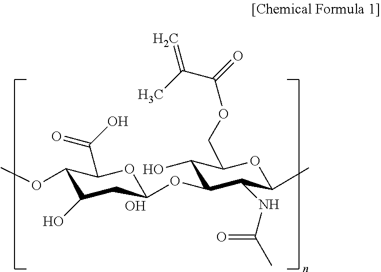

In the extracellular matrix-based hybrid ink for 3D printing according to an embodiment of the present disclosure, a chemical bond is formed between at least some of amine groups present in the extracellular matrix and the ethylenically unsaturated bond functional group present in the modified hyaluronic acid, and as a result, the extracellular matrix and the modified hyaluronic acid, which are the components of the hydride ink, are in a crosslinked state. The chemical bond between the amine group present in the extracellular matrix and the ethylenically unsaturated bond functional group present in the modified hyaluronic acid is formed by the aza-Michael addition reaction. The aza-Michael addition reaction is a nucleophilic conjugate addition reaction including N-nucleophiles and electrophiles such as activated alkenes, and in the present disclosure, the amine groups present in the extracellular matrix act as N-nucleophiles, and the ethylenically unsaturated bond functional groups present in the modified hyaluronic acid act as electrophiles.

In the extracellular matrix-based hybrid ink for 3D printing according to an embodiment of the present disclosure, a weight ratio of the extracellular matrix to the modified hyaluronic acid is preferably 1:0.01 to 1:0.8, and more preferably 1:0.05 to 1:0.5, when considering the chemical crosslinking density, which is a factor related to the mechanical properties, printability or drug release control of the hybrid ink. In addition, the concentration of the extracellular matrix in the hybrid ink is preferably 1 to 10% (w/v), and more preferably 2 to 8% (w/v), considering the mechanical properties, printability, or drug release control of the hybrid ink. In addition, the concentration of the modified hyaluronic acid in the hybrid ink is preferably 0.01 to 8% (w/v), and more preferably 0.1 to 4% (w/v), considering the mechanical properties, printability, or drug release control of the hybrid ink.

In addition, the extracellular matrix-based hybrid ink for 3D printing according to an embodiment of the present disclosure may preferably further include a growth factor for manufacturing a drug delivery patch, an angiogenesis-promoting patch, etc., to be described below. The growth factor may be selected from various known growth factors depending on a specific use, such as a drug delivery patch manufactured by 3D printing the hybrid ink, etc. For example, the growth factor may consist of at least one selected from the group consisting of vascular endothelial growth factor (VEGF), hepatocyte growth factor (HGF), epithermal growth factor (EGF), insulin-like growth factor (IGF), erythropoietin (EPO), fibroblast growth factor (FGF), brain-derived neurotrophic factor (BDNF), nerve growth factor (NGF), keratinocyte growth factor (KGF), interleukin (IL), colony-stimulating factor (CSF), angiopoietin (ANG), platelet-derived growth factor (PDGF), placental growth factor (PGF), transforming growth factor-α (TGF-α), transforming growth factor-β (TGF-β), matrix metalloproteinase (MMP), and bone morphogenetic protein (BMP). The growth factor concentration in the hybrid ink may be selected in various ranges depending on a specific use of the drug delivery patch manufactured by 3D printing the hybrid ink, etc., and for example, may be 0.001 to 1 g/l, and preferably 0.01 to 0.5 g/l when considering the drug delivery effect, etc.

A method for manufacturing an extracellular matrix-based hybrid ink for 3D printing according to an embodiment of the present disclosure includes preparing an extracellular matrix solution having a pH of 6.5 to 7.5; and adding and uniformly mixing a modified hyaluronic acid introduced with an ethylenically unsaturated bond functional group to the extracellular matrix solution, and then performing an aza-Michael addition reaction to obtain an ink in which the extracellular matrix and the modified hyaluronic acid are in a crosslinked state. In addition, the method for manufacturing the extracellular matrix-based hybrid ink for 3D printing according to an embodiment of the present disclosure may preferably further include adding and mixing a growth factor to the ink in order to use a patch formed by the ink as a drug delivery system or a disease therapeutic agent.

In the method for manufacturing the extracellular matrix-based hybrid ink for 3D printing according to an embodiment of the present disclosure, the detailed description of technical features of the extracellular matrix having the amine group, the modified hyaluronic acid introduced with the ethylenically unsaturated bond functional group, the aza-Michael addition reaction, the growth factor, etc. will be omitted by referring to the above-mentioned contents.

In the method for manufacturing the extracellular matrix-based hybrid ink for 3D printing according to an embodiment of the present disclosure, the preparing of the extracellular matrix solution having the pH of 6.5 to 7.5 consists of dispersing the extracellular matrix having the amine group in water and then adjusting the pH, or dissolving the extracellular matrix having the amine group in an acid solution and then neutralizing the solution with a base. The acid solution used to dissolve the extracellular matrix is not particularly limited to the type thereof, and is preferably a weak acid solution when considering applications such as biotransplantation. The weak acid may be selected from acetic acid, citric acid, butyric acid, palmitic acid, oxalic acid, tartaric acid, malic acid, succinic acid, and the like. In addition, the pH of the extracellular matrix solution is preferably close to neutral from the viewpoint of minimizing negative effects on the components constituting the ink and smoothly inducing the aza-Michael addition reaction, and may be selected from, for example, 6.8 to 7.4.

In the method for manufacturing the extracellular matrix-based hybrid ink for 3D printing according to an embodiment of the present disclosure, the extracellular matrix and the modified hyaluronic acid are present in a crosslinked state after the aza-Michael addition reaction, which is because a chemical bond is formed between at least some of the amine groups present in the extracellular matrix and the ethylenically unsaturated bond functional group present in the modified hyaluronic acid by the aza-Michael addition reaction. The aza-Michael addition reaction is performed at a temperature condition of 1 to 10° C., preferably 2 to 8° C., for 2 to 24 hours, preferably 6 to 18 hours, from the viewpoint of minimizing negative effects on the extracellular matrix and the modified hyaluronic acid and smoothly inducing the aza-Michael addition reaction.

In the method for manufacturing the extracellular matrix-based hybrid ink for 3D printing according to an embodiment of the present disclosure, the amount of modified hyaluronic acid added to the extracellular matrix solution is not significantly limited, but is preferably an amount of 1 to 80 parts by weight based on 100 parts by weight of the extracellular matrix, and more preferably 5 to 50 parts by weight based on 100 parts by weight of the extracellular matrix, when considering the mechanical properties, printability, or drug release control of the hybrid ink. In addition, the concentration of the extracellular matrix in the extracellular matrix solution is preferably 1 to 10% (w/v), and more preferably 2 to 8% (w/v), considering the mechanical properties, printability, or drug release control of the hybrid ink. In addition, the modified hyaluronic acid is added to the extracellular matrix solution preferably at a concentration of 0.01 to 8% (w/v), and more preferably at a concentration of 0.1 to 4% (w/v).

In the method for manufacturing the extracellular matrix-based hybrid ink for 3D printing according to an embodiment of the present disclosure, the amount of the growth factor added to the ink may be selected from various ranges depending on a specific use of the drug delivery patch manufactured by 3D printing the hybrid ink, etc. For example, the growth factor may be added to the ink at a concentration of 0.001 to 1 g/l, and may also be added at a concentration of 0.01 to 0.5 g/l when considering drug delivery effects, etc.

One aspect of the present disclosure relates to a biomaterial-based drug delivery patch having a multilayer structure in which drugs are spatiotemporally compartmentalized to sequentially sustained-release different drugs, and a method for manufacturing the same. In the biomaterial-based drug delivery patch according to an embodiment of the present disclosure and the method for manufacturing the same, the detailed description of technical features of the extracellular matrix having the amine group, the modified hyaluronic acid introduced with the ethylenically unsaturated bond functional group, the aza-Michael addition reaction, the growth factor, etc. will be omitted by referring to the above-mentioned contents.

The biomaterial-based drug delivery patch according to an embodiment of the present disclosure may be manufactured by 3D printing the extracellular matrix-based hybrid ink described above according to a predetermined design structure.

The biomaterial-based drug delivery patch according to one embodiment of the present disclosure is a multilayer patch including an inner core layer and an outer layer surrounding the inner core layer, and the inner core layer and the outer layer are made of the extracellular matrix-based hybrid ink. In addition, the biomaterial-based drug delivery patch exhibits rheological properties corresponding to a hydrogel state or a vitrified gel state.

In the biomaterial-based drug delivery patch according to one embodiment of the present disclosure, the hybrid ink includes an extracellular matrix having an amine group, a modified biopolymer introduced with an ethylenically unsaturated bond functional group, and a growth factor. The modified biopolymer may be preferably selected from a modified hyaluronic acid introduced with an ethylenically unsaturated bond functional group, a modified collagen introduced with an ethylenically unsaturated bond functional group, and the like. The ethylenically unsaturated bond functional group introduced into the modified biopolymer is not particularly limited to the type as long as it may perform an aza-Michael addition reaction with amine groups present in the extracellular matrix, and may be selected from, for example, a vinyl group, an allyl group, an acrylic group, a methacrylic group, etc. The modified hyaluronic acid may be selected from a methacrylated hyaluronic acid or an acrylated hyaluronic acid when considering biocompatibility, reactivity with amine groups, etc. In addition, the modified collagen may be selected from a methacrylated collagen or an acrylated collagen when considering biocompatibility, reactivity with amine groups, etc. The methacrylated collagen is a modified collagen in which a methacryl group is linked to an amine group present in collagen through a peptide bond, and the acrylated collage is a modified collagen in which an acrylic group is linked to an amine group present in collagen through a peptide bond. The structure and the manufacturing method of the methacrylated collagen or acrylated collagen are disclosed in various known literatures (e.g., U.S. Patent Publication No. 8658711; He Liang et al., Journal of Materials Chemistry B, 2018, 6, 3703-3715, etc.). The extracellular matrix and the modified biopolymer constituting the hybrid ink are present in a chemically crosslinked state. The chemical crosslinking of the extracellular matrix and the modified biopolymer results from a chemical bond formed between at least some of the amine groups present in the extracellular matrix and the ethylenically unsaturated bond functional group present in the modified hyaluronic acid by the aza-Michael addition reaction.

In addition, the extracellular matrix-based hybrid ink constituting the inner core layer and the extracellular matrix-based hybrid ink constituting the outer layer have different chemical crosslinking densities, and when considering a sequentially sustained release of the drug, it is preferred that the chemical crosslinking density of the extracellular matrix-based hybrid ink constituting the inner core layer is greater than the chemical crosslinking density of the extracellular matrix-based hybrid ink constituting the outer layer. The chemical crosslinking densities of the extracellular matrix-based hybrid ink constituting the inner core layer and the extracellular matrix-based hybrid ink constituting the outer layer may be controlled by the weight ratio of the extracellular matrix having the amine group and the modified biopolymer introduced with the ethylenically unsaturated bond functional group, which are the components of the extracellular matrix-based hybrid ink. The main components of the extracellular matrix-based hybrid ink is the extracellular matrix, but the chemical crosslinking densities of the main components of the extracellular matrix-based hybrid ink are increased by forming more chemical bonds by the aza-Michael addition reaction as the amount of the modified biopolymer to the extracellular matrix increases in the hybrid ink. For example, it is preferable that the weight ratio of the extracellular matrix to the modified biopolymer in the extracellular matrix-based hybrid ink constituting the inner core layer is 1:0.2 to 1:0.5. In addition, it is preferable that the weight ratio of the extracellular matrix to the modified biopolymer in the extracellular matrix-based hybrid ink constituting the outer core layer is 1:0.05 to 1:0.15. In addition, it is preferable that the concentration of the extracellular matrix in the extracellular matrix-based hybrid ink constituting the inner core layer and the extracellular matrix-based hybrid ink constituting the outer layer is 2 to 8% (w/v). It is preferable that the concentration of the modified biopolymer in the extracellular matrix-based hybrid ink constituting the inner core layer is 0.4 to 4% (w/v). In addition, it is preferable that the concentration of the modified biopolymer in the extracellular matrix-based hybrid ink constituting the outer layer is 0.1 to 1.2% (w/v).

In addition, the extracellular matrix-based hybrid ink constituting the inner core layer and the extracellular matrix-based hybrid ink constituting the outer layer include different growth factors. Accordingly, in the biomaterial-based drug delivery patch according to an embodiment of the present disclosure, the inner core layer and the outer layer contain different growth factors. The growth factor may be selected from various known growth factors depending on a specific use of the drug delivery patch. For example, the growth factor may be selected from vascular endothelial growth factor (VEGF) which is a growth factor inducing angiogenesis, hepatocyte growth factor (HGF), epithermal growth factor (EGF), fibroblast growth factor (FGF), angiopoietin (ANG), platelet-derived growth factor (PDGF), placental growth factor (PGF), transforming growth factor-α (TGF-α), transforming growth factor-β (TGF-β), matrix metalloproteinase (MMP), bone morphogenetic protein (BMP), and the like.

In the biomaterial-based drug delivery patch according to an embodiment of the present disclosure, when the extracellular matrix-based hybrid ink constituting the inner core layer has a higher chemical crosslinking density than the extracellular matrix-based hybrid ink constituting the outer layer, the growth factors contained in the inner core layer are not only spatially compartmentalized by a positional relationship with the growth factors contained in the outer layer, but also temporally compartmentalized by the chemical crosslinking density of the extracellular matrix-based hybrid ink, so that the growth factors contained in the inner core layer are slowly released in vivo at a slower rate than the growth factors contained in the outer layer. Considering the drug delivery effect of the multilayer structure patch, etc., it is preferable that the concentration of the growth factors in the extracellular matrix-based hybrid ink constituting the inner core layer and the extracellular matrix-based hybrid ink constituting the outer layer is 0.01 to 0.5 g/l.

A biomaterial-based drug delivery patch according to a preferred embodiment of the present disclosure is a multilayer patch in which a first layer, a second layer, and a third layer are sequentially laminated, and the second layer consists of a second inner layer located on the inner side and two second outer layers located on both outer sides, in which the first layer, the two second outer layers and the third layer are made of a first hybrid ink, and the second inner layer is made of a second hybrid ink. The first hybrid ink and the second hybrid ink include an extracellular matrix having an amine group, a modified hyaluronic acid introduced with an ethylenically unsaturated bond functional group, and a growth factor. The extracellular matrix and the modified hyaluronic acid constituting the first hybrid ink and the second hybrid ink are present in a chemically crosslinked state. In addition, the first hybrid ink and the second hybrid ink have different chemical crosslinking densities, and when considering sequential sustained-release of drugs, it is preferable that the chemical crosslinking density of the second hybrid ink is greater than the chemical crosslinking density of the first hybrid ink. In addition, the growth factors constituting the first hybrid ink and the second hybrid ink are selected from different growth factors.

A method for manufacturing a biomaterial-based drug delivery patch according to an embodiment of the present disclosure includes steps of preparing a first hybrid ink consisting of a mixture including an extracellular matrix having an amine group, a modified biopolymer introduced with an ethylenically unsaturated bond functional group, and a first growth factor, in which the extracellular matrix and the modified biopolymer are present in a chemically crosslinked state: preparing a second hybrid ink consisting of a mixture including an extracellular matrix having an amine group, a modified biopolymer introduced with an ethylenically unsaturated bond functional group, and a second growth factor different from the first growth factor, in which the extracellular matrix and the modified biopolymer are present in a chemically crosslinked state, and having a different chemical crosslinking density from the first hybrid ink: forming an inner core layer by 3D printing the second hybrid ink; and forming an outer layer surrounding the inner core layer by 3D printing the first hybrid ink and obtaining a multilayer structure. In the method for manufacturing the biomaterial-based drug delivery patch according to a preferred embodiment of the present disclosure, the multilayer structure consisting of the inner core layer and the outer layer surrounding the inner core layer formed by 3D printing the first hybrid ink and the second hybrid ink exists in an intermediate state between a sol and a hydrogel. The method for manufacturing the biomaterial-based drug delivery patch according to an embodiment of the present disclosure may further include obtaining a multilayer structure in a hydrogel state by heat-treating the multilayer structure at 25 to 45° C., preferably 30 to 40° C., for 0.5 to 3 hours, preferably 0.6 to 1.5 hours, in order to convert the multilayer structure into a hydrogel state. In addition, the method for manufacturing the biomaterial-based drug delivery patch according to an embodiment of the present disclosure may further include obtaining a vitrified multilayer structure by drying the multilayer structure in the hydrogel state under ventilated conditions for 6 to 24 hours, preferably 8 to 18 hours.

In the method for manufacturing the biomaterial-based drug delivery patch according to an embodiment of the present disclosure, it is preferable that the second hybrid ink has a higher crosslinking density than the first hybrid ink. In the method for manufacturing the biomaterial-based drug delivery patch according to an embodiment of the present disclosure, the first hybrid ink and second hybrid ink are extracellular matrix-based hybrid inks including the extracellular matrix as a main component. It is preferable that a weight ratio of the extracellular matrix to the modified biopolymer in the first hybrid ink is 1:0.05 to 1:0.15. In addition, the concentration of the extracellular matrix in the first hybrid ink is preferably 2 to 8% (w/v), and the concentration of the modified biopolymer is preferably 0.1 to 1.2% (w/v). It is preferable that a weight ratio of the extracellular matrix to the modified biopolymer in the second hybrid ink is 1:0.2 to 1:0.5. In addition, the concentration of the extracellular matrix in the second hybrid ink is preferably 2 to 8% (w/v), and the concentration of the modified biopolymer is preferably 0.4 to 4% (w/v).

A method for manufacturing a biomaterial-based drug delivery patch according to a preferred embodiment of the present disclosure includes steps of preparing a first hybrid ink consisting of a mixture including an extracellular matrix having an amine group, a modified hyaluronic acid introduced with an ethylenically unsaturated bond functional group, and a first growth factor, in which the extracellular matrix and the modified hyaluronic acid are present in a chemically crosslinked state; preparing a second hybrid ink consisting of a mixture including an extracellular matrix having an amine group, a modified hyaluronic acid introduced with an ethylenically unsaturated bond functional group, and a second growth factor different from the first growth factor, in which the extracellular matrix and the modified hyaluronic acid are present in a chemically crosslinked state, and having a different chemical crosslinking density from the first hybrid ink: forming a first layer by 3D printing the first hybrid ink: forming a second layer consisting of a second inner layer and two second outer layers by 3D printing the second hybrid ink on the inner side of the surface of the first layer and 3D printing the first hybrid ink on both outer sides of the surface of the first layer; and forming a third layer by 3D printing the first hybrid ink on the surface of the second layer and obtaining a multilayer structure. The method for manufacturing the biomaterial-based drug delivery patch according to a preferred embodiment of the present disclosure may further include obtaining a multilayer structure in a hydrogel state by heat-treating the multilayer structure including the first layer, the second layer and the third layer at 25 to 45° C., preferably 30 to 40° C., for 0.5 to 3 hours, preferably 0.6 to 1.5 hours. In addition, the method for manufacturing the biomaterial-based drug delivery patch according to a preferred embodiment of the present disclosure may further include obtaining a vitrified multilayer structure by drying the multilayer structure in the hydrogel state under ventilated conditions for 6 to 24 hours, preferably 8 to 18 hours. The first hybrid ink and the second hybrid ink have different chemical crosslinking densities, and when considering sequential sustained-release of drugs, it is preferable that the chemical crosslinking density of the second hybrid ink is greater than the chemical crosslinking density of the first hybrid ink. In addition, the first growth factor and the second growth factor constituting the first hybrid ink and the second hybrid ink are selected from different growth factors.

One aspect of the present disclosure relates to a biomaterial-based angiogenesis promoting patch having a multilayer structure in which drugs are spatiotemporally compartmentalized to sequentially sustained-release different angiogenesis promoting factors, and a method for manufacturing the same. In the biomaterial-based angiogenesis promoting patch and the method for manufacturing the same according to the present disclosure, as compared to the biomaterial-based drug delivery patch and the method for manufacturing the same described above, the remaining technical features are the same, except that the growth factors contained in each layer are specified as growth factors that promote an early stage of angiogenesis and growth factors that promote a mature stage of angiogenesis.

The biomaterial-based angiogenesis promoting patch according to an embodiment of the present disclosure is a patch having a multilayer structure including an inner core layer and an outer layer surrounding the inner core layer, and the outer layer is made of an extracellular matrix-based first hybrid ink, and the inner core layer is made of an extracellular matrix-based second hybrid ink. The first hybrid ink includes an extracellular matrix having an amine group, a modified biopolymer introduced with an ethylenically unsaturated bond functional group, and a first growth factor that promotes an early stage of angiogenesis, and the second hybrid ink includes an extracellular matrix having an amine group, a modified biopolymer introduced with an ethylenically unsaturated bond functional group, and a second growth factor that promotes a mature stage of angiogenesis. In addition, the extracellular matrix and the modified biopolymer constituting the first hybrid ink and the second hybrid ink exist in a chemically crosslinked state, and the second hybrid ink has a higher chemical crosslinking density than the first hybrid ink. In the biomaterial-based angiogenesis promoting patch according to a preferred embodiment of the present disclosure, the modified biopolymer introduced with the ethylenically unsaturated bond functional group is a modified hyaluronic acid introduced with an ethylenically unsaturated functional group. In addition, in the biomaterial-based angiogenesis promoting patch according to the present disclosure, the first growth factor that promotes the early stage of angiogenesis may be at least one selected from the group consisting of vascular endothelial growth factor (VEGF), angiopoietin (ANG), platelet-derived growth factor (PDGF), placental growth factor (PGF), epithermal growth factor (EGF), fibroblast growth factor (FGF), transforming growth factor-α (TGF-α), transforming growth factor-β (TGF-β), and matrix metalloproteinase (MMP). In addition, the second growth factor that promotes the mature stage of angiogenesis may be at least one selected from the group consisting of hepatocyte growth factor (HGF), bone morphogenetic protein (BMP), and transforming growth factor-β (TGF-β).

A biomaterial-based angiogenesis promoting patch according to a preferred embodiment of the present disclosure is a multilayer patch in which a first layer, a second layer, and a third layer are sequentially laminated, and the second layer consists of a second inner layer located on the inner side and two second outer layers located on both outer sides, in which the first layer, the two second outer layers and the third layer are made of a first hybrid ink, and the second inner layer is made of a second hybrid ink. The first hybrid ink includes an extracellular matrix having an amine group, a modified hyaluronic acid introduced with an ethylenically unsaturated bond functional group, and a first growth factor that promotes an early stage of angiogenesis. In addition, the second hybrid ink includes an extracellular matrix having an amine group, a modified hyaluronic acid introduced with an ethylenically unsaturated bond functional group, and a second growth factor that promotes a mature stage of angiogenesis. The extracellular matrix and the modified hyaluronic acid constituting the first hybrid ink and the second hybrid ink are present in a chemically crosslinked state. In addition, the first hybrid ink and the second hybrid ink have different chemical crosslinking densities, and when considering sequential sustained-release of drugs, it is preferable that the chemical crosslinking density of the second hybrid ink is greater than the chemical crosslinking density of the first hybrid ink.

A method for manufacturing a biomaterial-based angiogenesis promoting patch according to an embodiment of the present disclosure includes steps of preparing a first hybrid ink consisting of a mixture including an extracellular matrix having an amine group, a modified biopolymer introduced with an ethylenically unsaturated bond functional group, and a first growth factor that promotes an early stage of angiogenesis, in which the extracellular matrix and the modified biopolymer are present in a chemically crosslinked state: preparing a second hybrid ink consisting of a mixture including an extracellular matrix having an amine group, a modified biopolymer introduced with an ethylenically unsaturated bond functional group, and a second growth factor that promotes a mature stage of angiogenesis, in which the extracellular matrix and the modified biopolymer are present in a chemically crosslinked state, and having a higher chemical crosslinking density than the first hybrid ink: forming an inner core layer by 3D printing the second hybrid ink; and forming an outer layer surrounding the inner core layer by 3D printing the first hybrid ink and obtaining a multilayer structure. In addition, the method for manufacturing the biomaterial-based angiogenesis promoting patch according to an embodiment of the present disclosure may further include obtaining a multilayer structure in a hydrogel state by heat-treating the multilayer structure at 25 to 45° C., preferably 30 to 40° C., for 0.5 to 3 hours, preferably 0.6 to 1.5 hours, in order to convert the multilayer structure including the inner core layer and the outer layer into a hydrogel state. In addition, the method for manufacturing the biomaterial-based angiogenesis promoting patch according to an embodiment of the present disclosure may further include obtaining a vitrified multilayer structure by drying the multilayer structure in the hydrogel state under ventilated conditions for 6 to 24 hours, preferably 8 to 18 hours.

A method for manufacturing a biomaterial-based angiogenesis promoting patch according to a preferred embodiment of the present disclosure includes steps of preparing a first hybrid ink consisting of a mixture including an extracellular matrix having an amine group, a modified hyaluronic acid introduced with an ethylenically unsaturated bond functional group, and a first growth factor that promotes an early stage of angiogenesis, in which the extracellular matrix and the modified hyaluronic acid are present in a chemically crosslinked state: preparing a second hybrid ink consisting of a mixture including an extracellular matrix having an amine group, a modified hyaluronic acid introduced with an ethylenically unsaturated bond functional group, and a second growth factor that promotes a mature stage of angiogenesis, in which the extracellular matrix and the modified hyaluronic acid are present in a chemically crosslinked state, and having a higher chemical crosslinking density than the first hybrid ink: forming a first layer by 3D printing the first hybrid ink: forming a second layer consisting of a second inner layer and two second outer layers by 3D printing the second hybrid ink on the inner side of the surface of the first layer and 3D printing the first hybrid ink on both outer sides of the surface of the first layer; and forming a third layer by 3D printing the first hybrid ink on the surface of the second layer and obtaining a multilayer structure.

The biomaterial-based angiogenesis promoting patch according to the present disclosure may be used to prevent or treat angiogenesis-dependent diseases. Accordingly, the present disclosure provides a biomaterial-based patch for preventing or treating angiogenesis-dependent diseases.

The ‘angiogenesis-dependent diseases’ are diseases accompanied by symptoms in which blood supply is not smooth or angiogenesis is not properly performed. As used herein, the angiogenesis-dependent diseases are not particularly limited in types as long as they may be prevented or treated by promoting angiogenesis, and for example, the angiogenesis-dependent diseases may be at least one selected from the group consisting of ischemic disease, wounds, burns, psoriasis, chronic ulcers, myocardial infarction, angina pectoris, bedsores, alopecia, diabetic retinopathy, retinopathy of prematurity, age-related macular degeneration, glaucoma, diabetic foot ulcers, pulmonary hypertension, and cerebrovascular dementia. In addition, the ischemic disease may be at least one selected from the group consisting of cerebral ischemia, cardiac ischemia, diabetic vascular heart disease, heart failure, myocardial hypertrophy, retinal ischemia, ischemic colitis, ischemic acute renal failure, stroke, brain trauma, and neonatal hypoxia.

Hereinafter, the present disclosure will be described in more detail with reference to Examples. However, the following Examples are only for clearly illustrating the technical features of the present disclosure, but do not limit the protection scope of the present disclosure.

### 1. EXPERIMENT METHOD

### (1) Preparation of Vessel-Derived Decellularized Extracellular Matrix (VdECM) and Ink Containing the Same

Fresh porcine aortas purchased from a nearby slaughterhouse were chopped into cubes of approximately 2 mm in length and washed with deionized water for 6 hours to remove the remaining blood. Thereafter, the sliced aortic tissue was placed in a 0.3% (w/w) sodium dodecyl sulfate solution and stirred for 24 hours, and then placed in a 3% (w/w) Triton X-100 solution and stirred for 24 hours. Thereafter, the aortic tissue was rinsed with a phosphate buffered saline (PBS) solution for 24 hours to wash a chemical detergent, and then the aortic tissue was treated with a nuclease solution containing 75 U/ml DNase and 50 mM magnesium chloride (MgCl₂) in a PBS solution at 37° C. for 24 hours and decellularized. Thereafter, the decellularized tissue was treated and sterilized with a 0.1% peracetic acid solution dissolved in 4% ethanol for about 4 hours, and rinsed several times with deionized water and a PBS solution to obtain a vessel-derived decellularized extracellular matrix (VdECM). Thereafter, the vessel-derived decellularized extracellular matrix (VdECM) was rapidly frozen at −80° C. and freeze-dried for about 48 hours to obtain a powder form of vessel-derived decellularized extracellular matrix (VdECM).

100 mg of freeze-dried VdECM was dissolved in 7.5 mL of a 0.5 M acetic acid solution with 15 mg of pepsin by stirring at about 120 rpm, and the solution was filtered through a mesh filter with a pore size of 40 μm to remove undissolved particles, thereby obtaining a VdECM solution. Thereafter, a 10 N sodium hydroxide solution was added to the VdECM solution and stirred to neutralize the pH to 7.4, and a VdECM ink in a sol state was prepared.

### (2) Preparation of Hybrid Ink

VdECM and methacrylated hyaluronic acid (HAMA) were mixed in a neutral solution (pH 7), and then an aza-Michael addition reaction was performed at 4° C. for about 10 to 12 hours to newly synthesize a hybrid bioink in a sol state, which was named ‘HAVEM ink’. An acrylate functional group of the HAMA acted as a Michael acceptor, and an amine functional group present in the components of VdECM acted as a Michael donor, so that the aza-Michael addition reaction proceeded. The aza-Michael addition reaction was a nucleophilic conjugate addition reaction including N-nucleophiles and electrophiles such as activated alkenes. The aza-Michael addition reaction did not require a separate catalyst and was known to be the simplest and most efficient method for binding amines to α,β-unsaturated carbonyl compounds. The HAVEM ink was prepared into two types such as HAVEM ink (4V0.5H) and HAVEM ink (4V1H) by adding different amounts of VdECM and methacrylated hyaluronic acid (HAMA) to a neutral solution, and the compositions of components contained in the two types of HAVEM inks were summarized in Table 1 below. In addition, as a control, VdECM ink (4VOH) was prepared by adding only VdECM to a neutral solution (pH 7) at a concentration of 4% (w/v).

TABLE 1

| Classification of | Components and composition of HAVEM ink | |
|---|---|---|
| HAVEM ink | VdECM (%, w/v) | HAMA (%, w/v) |
| HAVEM ink (4V0.5H) | 4 | 0.5 |
| HAVEM ink (4V1H) | 4 | 1 |

In addition, in order to produce a spatiotemporal compartmentalized cerebral angiogenesis inducing (SCAI) patch, a vascular endothelial growth factor (VEGF) and a hepatocyte growth factor (HGF) were mixed with HAVEM ink (4V0.5H) or HAVEM ink (4V1H) at a concentration of 0.05 μg/μl, respectively, to prepare a total of four types of bioinks.

### (3) Manufacture of SCAI Patch

To develop a spatiotemporally separated dual growth factor release platform, a three-layered circular patch was manufactured using a 3D printing system. A first layer, a second outer layer, and a third layer were printed using HAVEM ink mixed with VEGF (recombinant human VEGF 165; PeproTech, USA). The second inner layer was printed using HAVEM ink mixed with HGF (recombinant human HGF; PeproTech, USA). Two types of HAVEM inks were loaded into a 3 ml syringe (Musashi Engineering Company, Japan) equipped with a 24 G plastic nozzle, respectively, and then loaded into a multihead 3D printing system (T&R Biofab, Korea). All the three layers were printed in a spiral pattern according to a programmed g-code at a temperature controlled at 4° C. After printing each layer, a pause time of 20 sec was given to prevent mixing the laminated layers. For gelation of the printed three-layered circular SCAI patch, the SCAI patch was placed in an incubator and treated under conditions of 5% carbon dioxide and 37° C. for 1 hour. Thereafter, the SCAI hydrogel patch was dried in a well-ventilated area for about 10 to 12 hours to be vitrified. Subsequently, the vitrified SCAI patch was used in subsequent cell and animal experiments.

### (4) Verification of Physicochemical Properties of Ink for 3D Printing and SCAI Patch

Rheological analysis of the biomaterial ink for 3D printing prepared above was performed using a 20 mm cone-plate type Discovery Hybrid Rheometer-2 (TA Instruments, USA) system. The analyzed ink for 3D printing was three types of neutralized inks (pH 7): 4% VdECM ink (4V0H): 4% VdECM ink containing 0.5% HAMA [HAVEM ink (4V0.5H)]; and 4% VdECM ink containing 1% HAMA [HAVEM ink (4V1H)]. Shear viscosity values were determined under a temperature condition of 4° C. and a shear rate condition increasing from 0.01 s⁻¹ to 1000 s⁻¹ to evaluate the flow behavior of each biomaterial ink. The gelation dynamic of each ink was measured under heating rate conditions of 5° C./min in a shear temperature change mode from 4° C. to 37° C. To determine the storage modulus and loss modulus of the biomaterial ink according to a frequency, a dynamic frequency change test was conducted on the ink gelled at 37° C. under 2% strain conditions and in the range of 0.1 to 100 rad/s. In addition, in order to confirm that VdECM and HAMA, which were components of the ink, were chemically crosslinked through an aza-Michael addition reaction mechanism, the freeze-dried ink was dissolved in D₂O and an ¹H NMR (400 MHz) spectrum was obtained using a Bruker Advance III HD 400 MHZ NMR spectrometer. Thereafter, the patch manufactured using the ink was vitrified, and then the tensile properties of the vitrified patch were confirmed using an MTS extensometer (AVX54, MTS). In order to prevent slippage of the vitrified patch during the tensile property test, a rectangular vitrified patch sample (25 mm×8 mm×400 μm) was prepared and fixed using screw grips mounted on the MTS extensometer.

### (5) Growth Factor Release Test

Recombinant human VEGF 165 (100-20; PeproTech, USA) and recombinant human HGF (100-39H; PeproTech, USA) were mixed into each ink to be a final concentration of 0.05 μg/μl, and then a multilayer patch was laminated using 3D printing, and a vitrified SCAI patch was manufactured through heat treatment and drying. The vitrified SCAI patch was subjected to double crosslinking, such as chemical crosslinking and thermal crosslinking. A vitrified circular disk-shaped SCAI patch (8 mm in diameter, 150 μm in height) was placed in 1.1 ml of a PBS solution at a temperature of 37° C. Thereafter, 1 ml of the solution was taken for each desired time point, stored at −80° C., and the same taken amount of PBS solution was added again. Thereafter, the amount of growth factors VEGF (DY293B; R&D Systems, USA) and HGF (DY294; R&D Systems, USA) released from each patch was quantified using an ELISA kit.

### (6) Cell Culture

Human brain microvascular endothelial cells (HBMECs: Innoprot, Korea) were inoculated in an endothelial cell medium (ECM: Innoprot, Korea) supplemented with 5% (v/v) fetal bovine serum, 1% (v/v) endothelial cell growth supplement, and 1% (v/v) penicillin/streptomycin, and cultured under conditions of 5% carbon dioxide and 37° C., while replacing the medium every 2 to 3 days. When confluence reached 90%, the cells were washed with Dulbecco's phosphate-buffered saline (DPBS), treated with a 0.25% Trypsin-EDTA (Gibco, USA) solution for 4 minutes under conditions of 5% carbon dioxide and 37° C., and then isolated and used the cell.

(7) rt-PCR

HBMEC cells were cultured at a density of 1.5×10⁴ per well in 1.5 mL of the medium using a 12-well plate. At this time, in an experimental group to examine the effect of the patch, the SCAI patch was placed on a transwell inserted into the well, and the effect of the growth factor released from the patch on the growth of HBMECs was confirmed. On days 3, 7, and 14 of culture, mRNA from cells in the wells was extracted using RNAiso plus (Takara Bio, Japan). The concentration and purity of the extracted RNA were measured using a Nanodrop Lite spectrophotometer (Thermo Fisher Scientific, USA). Thereafter, reverse transcription was performed on the extracted mRNA using the SuperScript IV First-Strand Synthesis System (Invitrogen Life Technologies, USA). Real-time PCR was performed using a StepOne Plus Real-Time Cycler (Applied Biosystems, USA) together with an SYBR-green I reagent (Takara, Japan). Data were analyzed by using StepOne software. v2.3, and the expression level of each gene normalized to GAPDH expression was calculated according to a 2^{-ΔΔCT} method. The primer sequences used for real-time PCR were designed based on the gene sequences published in NCBI and PubMed, and the primer sequences used for real-time PCR were summarized in Table 2 below.

TABLE 2

| Target | Description of | Base sequence of primer |
|---|---|---|
| gene | primer | (5′→3′) |
| hu- | Forward primer | 5′-CAATGACCCCTTCATTGACC-3′ |
| GAPDH | Reverse primer | 5′-GACAAGCTTCCCGTTCTCAG-3′ |
| hu- | Forward primer | 5′-CACCCAGCACATCATGCAA-3′ |
| FLT1 | Reverse primer | 5′-TTCCCCCCTGCATTGGA-3′ |
| hu- | Forward primer | 5′-ATTCCTCCCCCGCATCA-3′ |
| FLK1 | Reverse primer | 5′-GCTCGTTGGCGCACTCTT-3′ |
| hu- | Forward primer | 5′-GCGGGACAAAACCATGGA-3′ |
| ANGPT1 | Reverse primer | 5′-AACTGGGCCCTTTGAAGTAGTG-3′ |
| hu- | Forward primer | 5′-TATGATGCCCAGTTTGAGGT-3′ |
| PECAM | Reverse primer | 5′-GAATACCGCAGGATCATTTG-3′ |

### (8) Evaluation of Effect of SCAI Patch on Cell Experiment

To evaluate in vitro cytotoxicity, a vitrified SCAI patch was placed on a transwell inserted into each well, and HBMECs were added to the bottom of each well at a density of 2×10⁴ cells/ml. Thereafter, the cells were cultured for 7 days under conditions of 5% carbon dioxide and 37° C. and then stained using a live/dead assay kit (LIVE/DEAD Cell Viability Assay, Thermo Fisher Scientific, USA). Live cells and dead cells were labeled with green and red fluorescent substances, respectively, and observed under a fluorescence microscope (Nikon Instruments, Japan). Cell proliferation was quantified using a Cell Counting Kit-8 Assay Kit (Dojindo Molecular Technologies, Japan). For each experimental group with a patch and a control group without a patch, cells were cultured until a specific time and then cultured with a CCK-8 solution diluted 1:10 in a fresh medium at 37° C. for 3 hours. Thereafter, the treated solution was transferred to a transparent 96-well plate and the absorbance at 450 nm was measured using a Multiskan™ GO Microplate Spectrophotometer (Thermo Fisher Scientific, USA).

### (9) Evaluation of Effect of SCAI Patch on Animal Experiment

All animal studies were performed in accordance with national regulatory guidelines after receiving approval from the local Institutional Animal Care and Use Committee (POSTECH-2021-0022). Eight-week-old male Sprague-Dawley (SD) rats weighing 200 to 300 g were used for an in vivo experiment. Anesthesia of rats was initially performed using an anesthesia induction chamber containing 4.0% isoflurane and then maintained at a concentration of 1.5% isoflurane during surgery. The head was fixed to a stereotaxic frame with an ear bar, and a body temperature was maintained with a heating pad. The head was shaved, and then the skin of the hemisphere was incised. The remaining epidermis and debris were removed with a saline solution. Then, a craniotomy was carefully performed using a dental drill. The exposed brain tissue was completely covered with a polydimethylsiloxane (PDMS) membrane having a size of 6 mm×11 mm and a thickness of 150 to 180 μm, sterilized, and then immersed in a saline solution. The boundary of the PDMS membrane was bonded to the skull using a cyanoacrylate adhesive (Loctite, USA). Thereafter, a dental resin (OA2: Denkist Inc., Korea) was applied along the edge of the PDMS membrane and irradiated with ultraviolet light for 15 seconds to be semi-permanently bonded to the skull. Finally, the rats were administered with Baytril (5 mg/kg: Bayer, Germany) subcutaneously and returned to the cages. Then, Tylenol (0.5 mg/mL: Janssen, Korea) and Marboxyl (0.06 mg/mL: Vetoquinol, Korea) were continuously supplied through drinking water to relieve inflammation. Two weeks after surgery, the brain tissue was collected and analyzed using hematoxylin/eosin (H&E) staining and immunohistochemistry (IHC), and then the functional angiogenesis efficacy of the SCAI patch was evaluated based on the results.

### (10) Photoacoustic Monitoring of Cerebral Angiogenesis Pattern Induced by SCAI Patch

An optical resolution photoacoustic microscopy (OR-PAM) system (OptchoM, Optcho, Korea) was used to monitor cerebral angiogenesis. The OR-PAM was a bioimaging tool for noninvasive exploration of volumetric microvasculature, which generated ultrasound in vessels using a nanopulse laser system (VPFL-G-10, Spectra-Physics, USA) with a wavelength of 532 nm. The ultrasound generated by the system was measured by a customized ultrasonic transducer at a center frequency of 20 MHz and a bandwidth of 60%. The segmented images may be combined to reconstruct a wide field of view (FOV) image. The segmented images were acquired using a galvanometer scanner (GVS001; Thorlabs, USA) and two linear motorized stages (L-509.10SD00; Physik Instrumente, Germany). The galvanometer scanner adjusted laser pulses with a scan range length of 1.6 mm (400 pixels) and a scan speed of 50 Hz. A linear motorized stage with a maximum scan range of 26 mm (step size of 5 μm) moved the scan portion of the OR-PAM system in a direction perpendicular to the scan direction of the galvanometer scanner. The scan range of the stage was adjusted according to the size of ROI. Another linear stage moved the scan portion in the same scan direction as the galvanometer scanner to provide the segmented images in another area. The acquired segmented images were reconstructed into wide-angle FOV images by a structural similarity (SSIM)-based volumetric image registration algorithm using pyramid blending. In order to calculate the density of newly generated vessels with a diameter less than 50 μm, a mask removing an outer area of the ROI was first formed, and then vessels with a diameter greater than about 150 μm were removed from PA maximum amplitude projection (MAP) images of the rat brain. The area of the ROI was calculated as the area of the mask. Thereafter, a Hessian-based vessel filter was applied, vessels with a diameter of 50 μm or more were extracted from the filtered image, and a large vessel map was created based on the extracted vessels. Finally, the large vessel map was removed from the vessel filtering image and a small vessel map was reconstructed with small vessels with a diameter of less than 50 μm, and the area of the small vessels was calculated based on the small vessel map.

### (11) Histological Analysis

The brain was extracted, a tissue sample was fixed in a 4% paraformaldehyde solution, and a surgical site attached with the patch was incised. Thereafter, the tissue sample was solidified in paraffin, and the paraffin-embedded tissue sample was sectioned transversely to a thickness of less than 5 μm for a H&E staining experiment. The stained images were imaged using a microscope system (Leica DM750; Leica).

### (12) Immunohistochemistry

A tissue sample fixed in a 4% paraformaldehyde solution was solidified in paraffin and sectioned at a 4 μm thickness for immunofluorescence staining. Thereafter, deparaffinization and rehydration steps were performed using a buffer solution. Thereafter, the sections were placed in a 10% hydrogen peroxide solution, cultured at room temperature for 10 min, and then stained with a primary antibody against CD31 (1:1000; abcam, UK). The stained area was further cultured with a peroxidase-conjugated secondary antibody (Envision+Rabbit; DAKO, USA) for 30 min, and then counterstained with Mayer' Hematoxylin. The obtained slice was imaged using a microscope system (Leica DM750; Leica).

### (13) Statistical Analysis

All statistical analyses were performed using the GraphPad Prism 8.2.1 program (GraphPad Software, USA). The significance of differences between data was evaluated using two-way ANOVA or t-test. The expression for statistical significance was as follows: *p<0.05, **p<0.01, ***p<0.001, ****p<0.0001.

### 2. EXPERIMENT RESULT

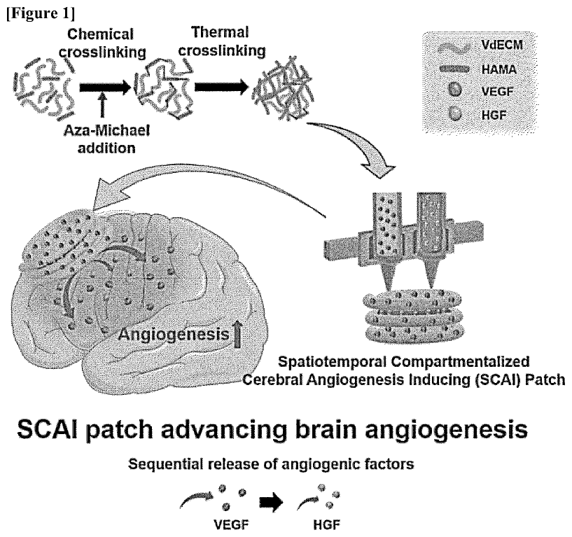

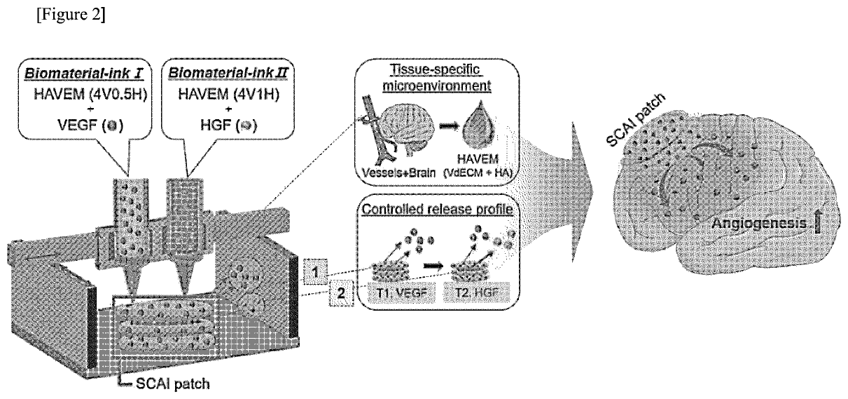

FIG. 1 illustrates a schematic process of manufacturing a hybrid ink (HAVEM ink) from vessel-derived decellularized extracellular matrix (VdECM) and methacrylated hyaluronic acid (HAMA) using a dual crosslinking mechanism such as chemical crosslinking and thermal crosslinking in the present study. FIG. 2 illustrates a schematic process of manufacturing a spatiotemporal compartmentalized cerebral angiogenesis inducing (SCAI) patch using a 3D printing process by adding VEGF and HGF, which are cerebral angiogenesis inducing growth factors, to hybrid inks with different crosslinking densities, respectively, and a schematic process of sequentially releasing the VEGF and HGF from the SCAI patch, in the present study.

As shown in FIGS. 1 and 2, the present inventors manufactured a sophisticated SCAI hydrogel patch in which two angiogenesis growth factors were compartmentalized and sequentially sustained-released by using extracellular matrix (ECM)-based hybrid ink and 3D printing technology. A hybrid ink was formed by binding vessel-derived decellularized extracellular matrix (VdECM) and methacrylated hyaluronic acid (HAMA) through a chemical crosslinking method called the aza-Michael addition reaction. The aza-Michael addition reaction was a very ideal mechanism for manufacturing the SCAI patch because the release profile and printability of angiogenic factors may be controlled simply by changing only the chemical bond density without requiring an exogenous catalyst. The present inventors placed different growth factors inside and outside the SCAI patch and spatially separated the growth factors in order to sequentially release the growth factors after transplanting the patch and used two types of hybrid inks (HAVEM inks) with different chemical crosslinking densities to load different growth factors. The SCAI patch was manufactured by 3D printing the two types of hybrid inks (HAVEM inks) loaded with the different growth factors and then a vitrification process was applied to the SCAI patch to manufacture a soft, flexible, and easy-to-manipulate brain transplantable patch. It was demonstrated in vitro and in vivo that the patch manufactured above had a therapeutic effect of promoting angiogenesis. In particular, the present inventors observed the angiogenesis pattern of cerebral microvessels over time for 14 days after patch transplantation using a photoacoustic microscope applied with a non-labeled method. According to quantified results, it was shown that the SCAI patch showed significant angiogenesis performance in the brain area.

### (1) Physicochemical Properties of Hybrid Ink (HAVEM Ink)

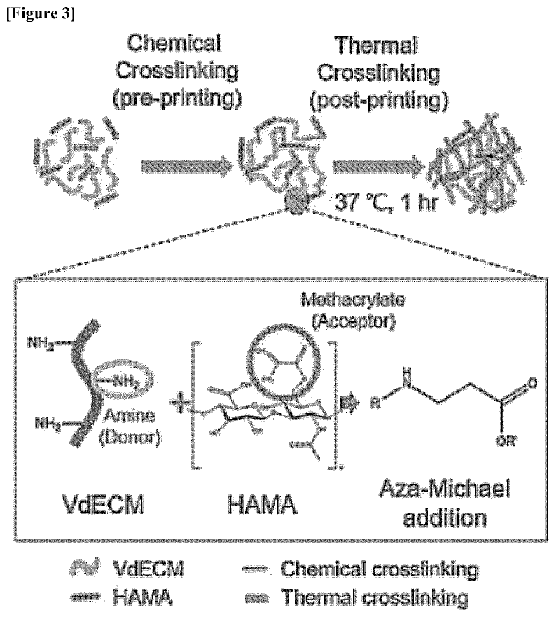

In order to load and release angiogenic factors from the patch at the desired amount and speed, a novel biomaterial-based dual-crosslinking hybrid ink was manufactured from HAMA and VdECM, which was named an ‘HAVEM ink’. FIG. 3 schematically illustrates a process of manufacturing an HAVEM ink from vessel-derived decellularized extracellular matrix (VdECM) and methacrylated hyaluronic acid (HAMA) through an aza-Michael addition reaction. First, in the chemical crosslinking process of the HAVEM ink, the methacrylate group of HAMA and the amine group of VdECM were bound through the aza-Michael addition reaction. The chemical crosslinking density could be controlled by adjusting the ratio of each VdECM and HAMA.

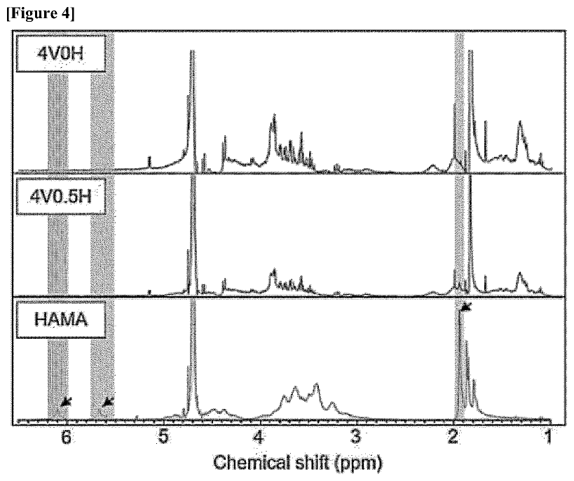

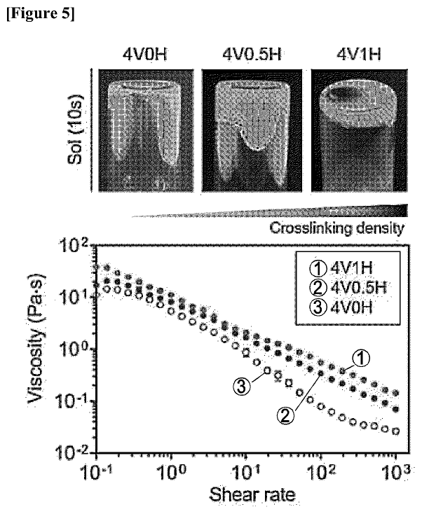

At this time, the chemical bond formed through the aza-Michael addition reaction was confirmed through NMR spectroscopy. FIG. 4 illustrates spectra measured by nuclear magnetic resonance (NMR) with respect to a VdECM ink (4V0H), HAMA, and an HAVEM ink (4V0.5H) to demonstrate the occurrence of crosslinking between vessel-derived decellularized extracellular matrix (VdECM) and methacrylated hyaluronic acid (HAMA). NMR characteristic peaks associated with the methacrylate functional group were highlighted at 1.9, 5.7, and 6.1 ppm.

In the NMR spectrum results of HAMA, peaks at 1.9, 5.7, and 6.1 ppm, were observed as the NMR characteristic peaks of the methacrylate group contained in HAMA, but in the spectrum of an ink (4V0H) containing only VdECM, these peaks were not observed at the same positions, which indicated that there was no methacrylate group. In addition, the NMR characteristic peaks corresponding to the methacrylate group could not be observed even in an HAVEM ink group (4V0.5H and 4V1H). This means that all the methacrylate groups of HAMA successfully reacted with the amine groups of VdECM to form chemical crosslinking.

Thereafter, a change in printability according to a ratio of HAMA in the HAVEM ink was confirmed. An upper photograph of FIG. 5 shows sol-state images of VdECM ink (4V0H), HAVEM ink (4V0.5H), and HAVEM ink (4V1H) according to a difference in chemical crosslinking density, and a lower graph of FIG. 5 shows a result of measuring viscosities of VdECM ink (4V0H), HAVEM ink (4V0.5H), and HAVEM ink (4V1H) under printing conditions of 4° C. and various shear rates. As shown in FIG. 5, both HAVEM inks (4V0.5H and 4V1H) with different chemical crosslinking densities had slightly higher viscosities and exhibited rheologically shear thinning properties than the ink (4V0H) containing only VdECM. More specifically, as the chemical crosslinking density increased, the fluidity of the ink decreased and the viscosity increased. Meanwhile, the viscosity of each HAVEM ink was hardly affected over time.

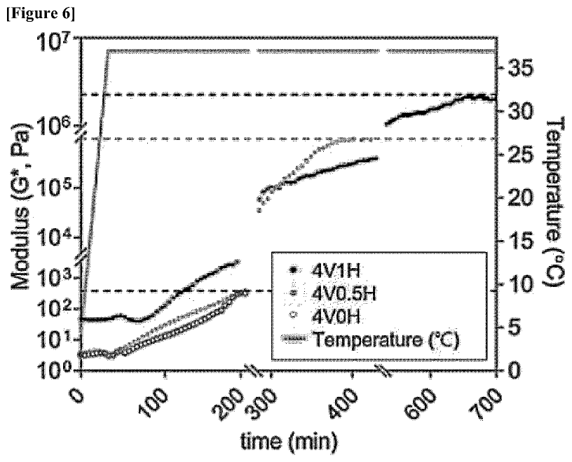

In order to determine the effect of the controlled crosslinking density on the mechanical properties of the HAVEM ink, a complex modulus change of the prepared ink was monitored while increasing the temperature of the HAVEM ink from 4° C. to 37° C. FIG. 6 illustrates changes in elastic modulus of VdECM ink (4V0H), HAVEM ink (4V0.5H), and HAVEM ink (4V1H) according to a temperature change from 4° C. to 37° C. The gelation dynamics of inks may be determined by a change in elasticity in FIG. 6. As shown in FIG. 6, the HAVEM ink (4V1H) containing a higher HAMA concentration showed a trend of a greater increase in complex modulus as thermal crosslinking progressed when compared to the HAVEM ink (4V0.5H) containing a lower HAMA concentration and the VdECM ink (4VOH) containing VdECM alone. In addition, the time required to reach an equilibrium was observed to be relatively longer in HAVEM ink (4V1H) with a higher chemical crosslinking density than in other inks (4V0.5H, 4V0H), which was inferred to be because as the proportion of HAMA in the ink increased, more chemical crosslinking occurred, resulting in a higher complex modulus. At an equilibrium state, the complex modulus of HAVEM ink (4V1H) was observed to be 1.91 times and 3,045 times greater than those of HAVEM ink (4V0.5H) and VdECM ink (4VOH).

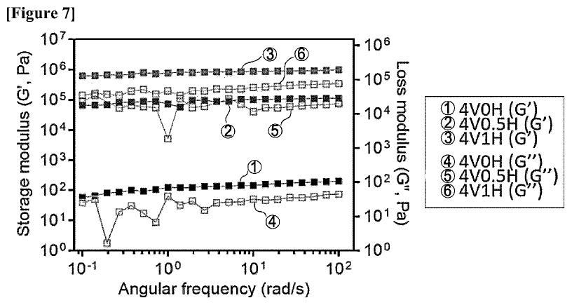

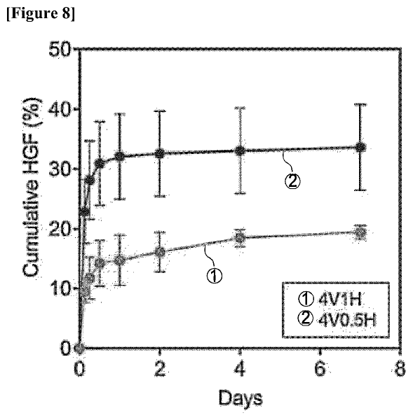

Next, when the complex moduli of the inks in the gel state where chemical crosslinking and thermal crosslinking were completed were confirmed, HAVEM (4V1H) ink showed a higher complex modulus value than VdECM (4V0H) ink and HAVEM (4V0.5H) ink. FIG. 7 illustrates storage modulus and loss modulus of VdECM ink (4V0H), HAVEM ink (4V0.5H), and HAVEM ink (4V1H) according to a change in frequency at a temperature of 37° C. As shown in FIG. 7, it was confirmed that the hybrid ink had higher structural fidelity as the crosslinking ratio increased during the printing process. By varying the chemical crosslinking density of the HAVEM ink, the hybrid ink was able to be induced to have appropriate mechanical properties, which directly affected the control of the release pattern of the angiogenic factor. FIG. 8 shows a result of measuring release profiles of HGF encapsulated in HAVEM ink (4V0.5H) and HAVEM ink (4V1H) for 7 days. As shown in FIG. 8, it was confirmed that the HGF release from HAVEM ink (4V1H) containing HGF was much slower than the HGF release from HAVEM ink (4V0.5H) containing HGF.

### (2) Design and Manufacture of SCAI Patch Using 3D Printing Technology

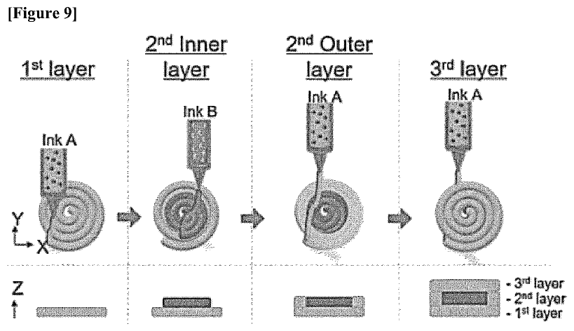

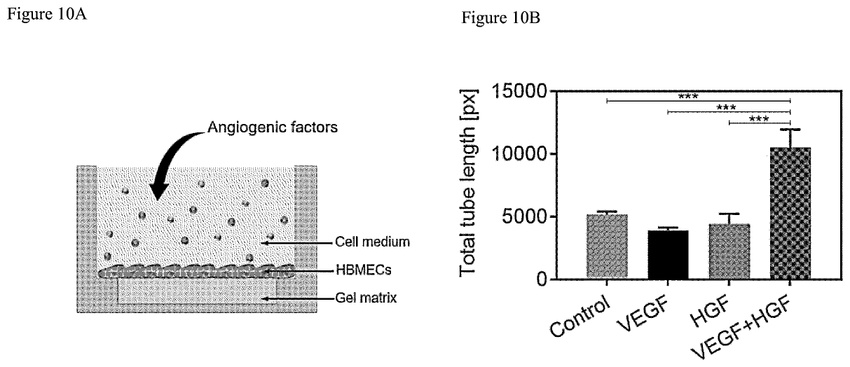

Two types of HAVEM inks such as HAVEM ink (4V0.5H) and HAVEM ink (4V1H) were used to manufacture an SCAI patch. FIG. 9 schematically illustrates a process of manufacturing an SCAI patch using 3D printing technology in the present study. As shown in FIG. 9, the patch structure was designed in a circular shape with a total of three layers, distinguished by an outer layer and an inner layer, and 3D printed. The patch structure was designed so that the outer layer was loaded with VEGF (angiogenic factor involved in the early stage of angiogenesis) to be released early, and the inner layer was applied with HGF (angiogenic factor involved in late angiogenesis) to be delayed-released. FIG. 10 shows a result of measuring an angiogenesis effect of various angiogenic factors by a tube formation assay. FIG. 10A is a schematic diagram of a tube formation assay, and FIG. 10B shows total tube lengths measured after 48 hours for each of an untreated group (Control), and samples treated with VEGF, HGF, and VEGF and HGF in combination. As shown in FIG. 10, the combination of growth factors effectively induced rapid and extended tube formation in human brain microvascular endothelial cells (HBMECs), and formed the highest total tube length after 48 hours compared to the groups treated with only one type of growth factor. Based on these results, a circular SCAI patch with a diameter of 8 mm was manufactured using HAVEM ink (4V0.5H; ink A) containing VEGF and HAVEM ink (4V1H; ink B) containing HGF. First, ink A was printed along a spiral trajectory with a diameter of 8 mm to form a first layer. To physically block the HGF from the outside, ink B was printed along a spiral trajectory with a diameter of 6 mm at the inner location of the first layer to form a second inner layer, and then the second outer layer and a third layer were printed using ink A under the same printing conditions. Each layer was printed and then paused for 20 seconds, and a Z-axis was raised by 200 μm and the next layer was printed. As a result, HGF could not only be spatially compartmentalized through the printing process, but also temporally compartmentalized using HAVEM ink (4V1H) as an ink containing HGF, thereby preventing HGF from being released from the patch during the early stage of angiogenesis. In the present study, the inner core layer was printed with high-crosslinking density HAVEM ink (4V1H: ink B) containing HGF, and the outer layer completely surrounding the core layer was printed with low-crosslinking density HAVEM ink (4V0.5H: ink A) containing VEGF. Meanwhile, since the first layer, the second outer layer, and the third layer of the patch were all formed of HAVEM ink (4V0.5H; ink A) containing VEGF, VEGF may be released immediately after the patch was transplanted to amplify the effect on the early angiogenesis process.

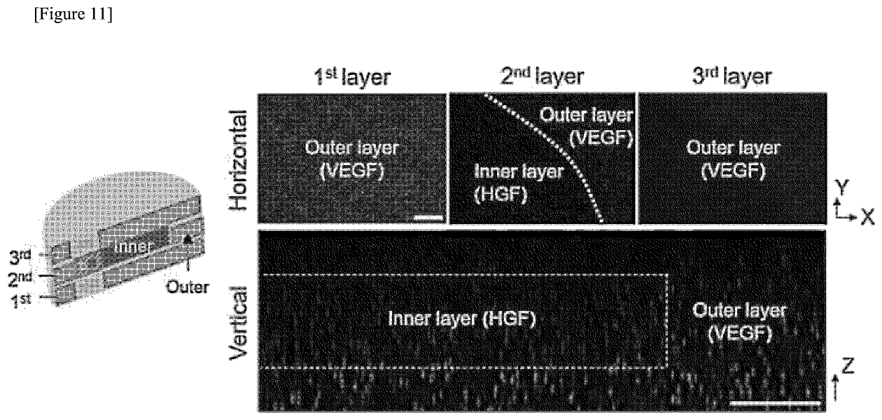

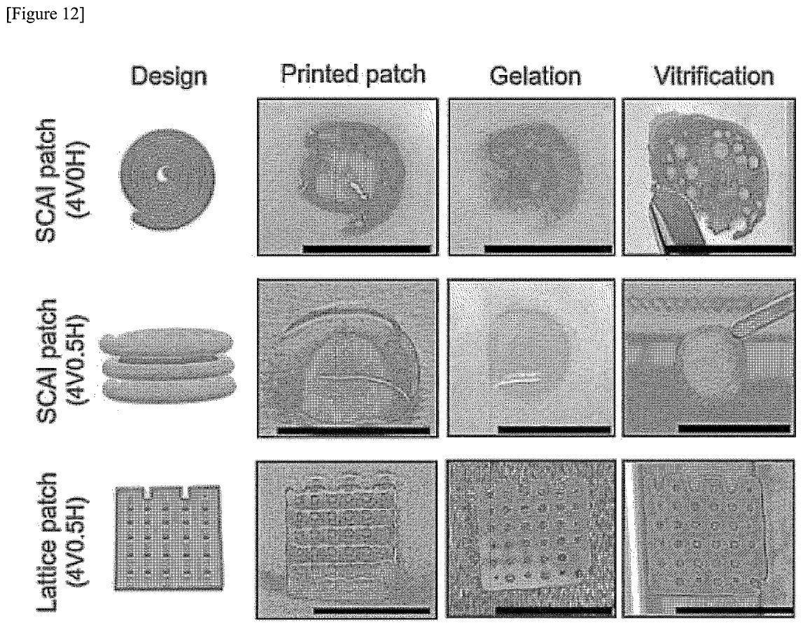

FIG. 11 shows images of a cross-sectional structure and each cross-section of the SCAI patch manufactured in the present study photographed using a confocal microscope (scale bars: 200 μm). FIG. 12 shows images of design structures of SCAI patches manufactured with various combinations of HAVEM inks and images of respective patches in printing, gelation, and vitrification steps. In FIG. 12, a patch of Design structure 1 (leftmost column) was manufactured by printing in a circular shape with VdECM ink (4VOH), a patch of Design structure 2 (intermediate column) was manufactured by printing an inner core layer with HAVEM ink (4V1H) and printing an outer layer surrounding the inner core layer with HAVEM ink (4V0.5H), and a patch of Design structure 3 (rightmost column) was manufactured by printing in a lattice shape using HAVEM ink (4V0.5H) (scale bars: 10 mm). As shown in FIGS. 11 and 12, the compartmentalized structure of the patch was well maintained even after the patch was printed and then subjected to a thermal cross-linking process. This demonstrates that the HAVEM ink has excellent printability to easily print a desired structure even using multimaterial-based printing processes. Actually, since it was relatively difficult to implement a precise shape when printed with VdECM ink (4V0H) compared to HAVEM ink (4V0.5H), it was not easy to be printed in the desired SCAI patch shape. In contrast, it was confirmed that HAVEM ink (4V0.5H) was able to print not only a compartmentalized SCAI patch structure but also a lattice-shaped hydrogel structure with a double-layer microporous structure. Therefore, when using the HAVEM ink prepared in the present study, it is possible to manufacture patches of various structures.

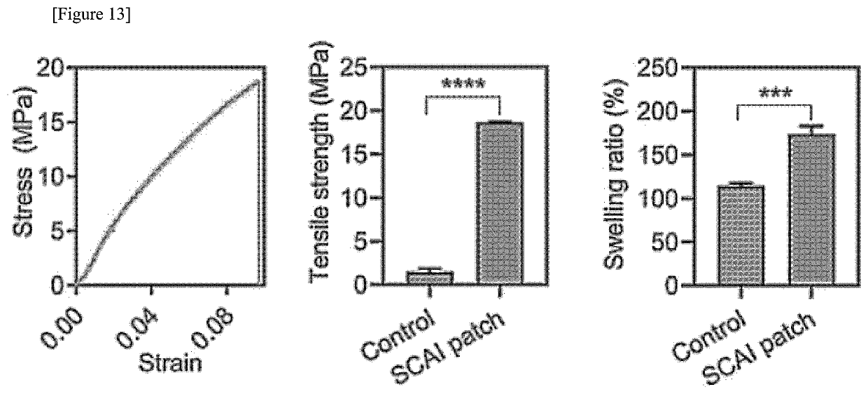

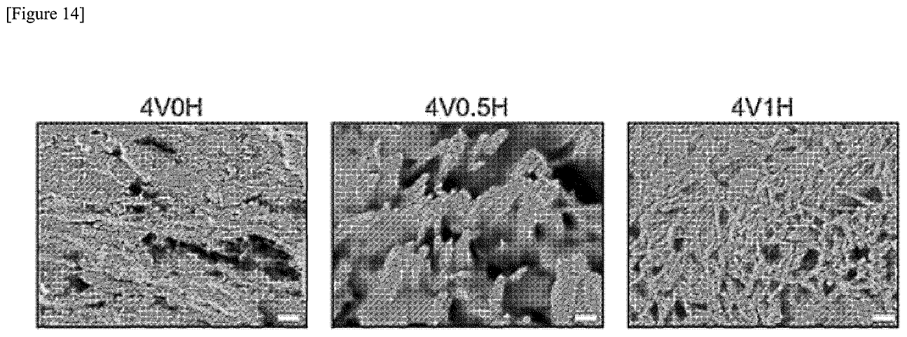

The inner core layer was printed with HAVEM ink (4V1H), and the outer layer surrounding the inner core layer was printed with HAVEM ink (4V0.5H) to be formed into an SCAI patch, and then the SCAI patch was thermally crosslinked through a specified heat treatment and then vitrified so as to facilitate transplantation, handling, and storage during surgery. To evaluate the mechanical properties of the SCAI patch subjected to the vitrification process, tensile stress-strain characteristics were investigated. FIG. 13 shows tensile strength measurement results and swelling ratios of the SCAI patch through the vitrification process manufactured in the present study. In FIG. 13, “Control” represents an SCAI patch that was not subjected to the vitrification process, and “SCAI patch” represents an SCAI patch that was subjected to the vitrification process. As shown in FIG. 13, the vitrified SCAI patch exhibited a tensile strength of 18.62 MPa, which was 12.46 times higher than that of the control sample that was not subjected to the vitrification process. In addition, the vitrified patch swelled up to 151% by rapidly reabsorbing water when immersed in saline solution. The microstructure of the vitrified SCAI patch was imaged using a scanning electron microscope (SEM), and the interconnected structure and the porous structure were confirmed to vary depending on a crosslinking density. FIG. 14 shows SEM photographed images of various vitrified patches manufactured in the present study (scale bars: 1 μm). In FIG. 14, “4VOH” represents a vitrified patch made of VdECM ink (4VOH), “4V0.5H” represents a vitrified patch made of HAVEM ink (4V0.5H), and “4V1H” represents a vitrified patch made of HAVEM ink (4V1H). As shown in FIG. 14, as the crosslinking density increased, the density of the fiber form also increased, but as the crosslinking density increased in the order of 4V0H, 4V0.5H, and 4V1H in each structure, it was confirmed that the density of the fiber form in the SEM image also increased.

### (3) In Vitro Experiment for Evaluating Continuous and Sequential Release Profile and Cell Behavior of SCAI Patch

The present inventors established the characteristics of the SCAI patch and then confirmed the release pattern of growth factors from the manufactured patch. To sequentially sustained-release VEGF and HGF, two strategies were utilized. By controlling the chemical crosslinking density of HAVEM ink containing each growth factor and using 3D printing technology, a hydrogel patch was manufactured so that two angiogenic factors were spatiotemporally compartmentalized.

Hydrogels with a lower chemical crosslinking density contained more water and thus degraded more quickly than hydrogels with a higher chemical crosslinking density. According to this principle, the higher the chemical crosslinking density in the hydrogel, the lower the release rate of the growth factors encapsulated in the hydrogel. Since the SCAI patch of the present disclosure was a chemically crosslinked hydrophilic matrix, the degradation rate may be controlled by adjusting the density of chemical bonds, thereby controlling the release pattern of growth factors loaded into the corresponding hydrogel.

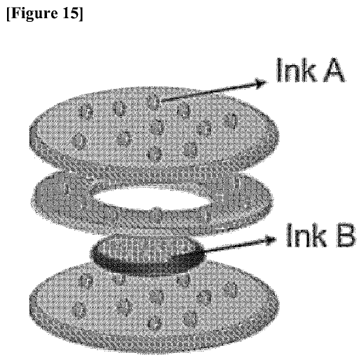

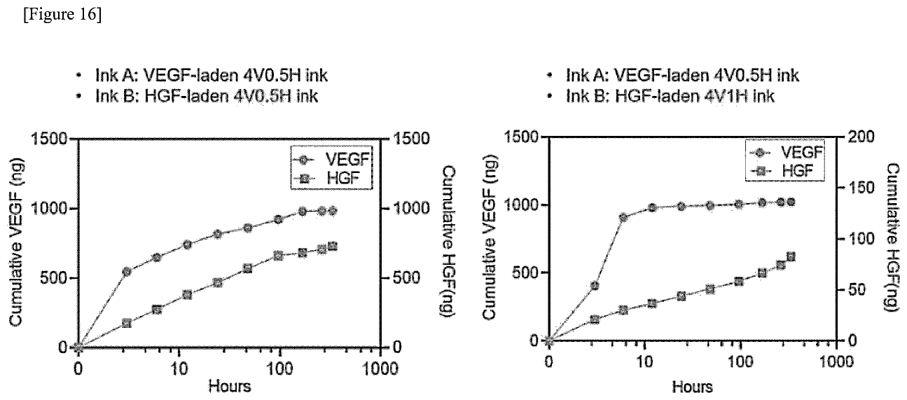

To validate the strategy designed for sequential drug sustained-release of VEGF and HGF, two types of multilayer SCAI patches were manufactured and the release patterns of growth factors were confirmed for two experimental groups. FIG. 15 schematically shows a structure of a multilayer SCAI patch designed in the present study for sequential drug sustained-release of VEGF and HGF. As shown in FIG. 15, the multilayer SCAI patch designed in the present study consisted of an inner core layer made of a hybrid ink (ink B) containing HGF and an outer layer made of a hybrid ink (ink A) containing VEGF and surrounding the inner core layer. FIG. 16 shows a result of evaluating cumulative release profiles of VEGF and HGF for one month with respect to two types of multilayer SCAI patches designed in the present study for sequential drug sustained-release of VEGF and HGF, respectively. A left experimental group of FIG. 16 shows results of observing drug sustained-release and sequential release patterns on a multilayer SCAI patch in which the ink conditions are the same and the drugs are just spatially compartmentalized by consisting of an outer layer formed using HAVEM ink (4V0.5H) containing VEGF at a content of 0.05 μg/μl as ink A and an inner core layer formed using HAVEM ink (4V0.5H) containing HGF at a content of 0.05 μg/μl as ink B. A right experimental group of FIG. 16 shows results of observing drug sustained-release and sequential release patterns on a multilayer SCAI patch in which the drugs are spatiotemporally compartmentalized by consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B. As shown in FIG. 16, it was confirmed that VEGF loaded on the outer layer of the SCAI patch was released within one day (24 hours) at 50.07%+10.63% in both groups without a particular difference. Meanwhile, in the case of HGF loaded on the inner core layer of the SCAI patch, since the HGF was located in the inner layer that could be relatively spatially protected from water erosion in both groups, a sustained-release pattern was shown compared to VEGF. Furthermore, as shown in the right experimental group, by controlling the chemical crosslinking density of HAVEM ink, it was confirmed that when there was a difference between external and internal conditions even in a material portion, the sustained release and sequential release patterns were more distinctly shown than when only the spatial compartment was implemented. In the right experimental group where HGF was loaded on HAVEM ink (4V1H) different from the outer layer by controlling the chemical crosslinking density of the outer layer and the inner layer, it took 30 days to release approximately 50% of the initial HGF loading. On the contrary, in the left experimental group where HGF was loaded with the same HAVEM ink (4V0.5H) as the outer layer without controlling the chemical crosslinking density of the outer and inner layers, it took about 4 days to release HGF in a similar amount to that in the right experimental group.

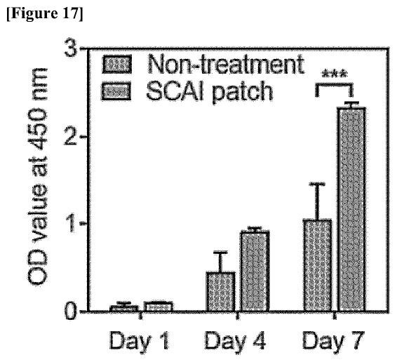

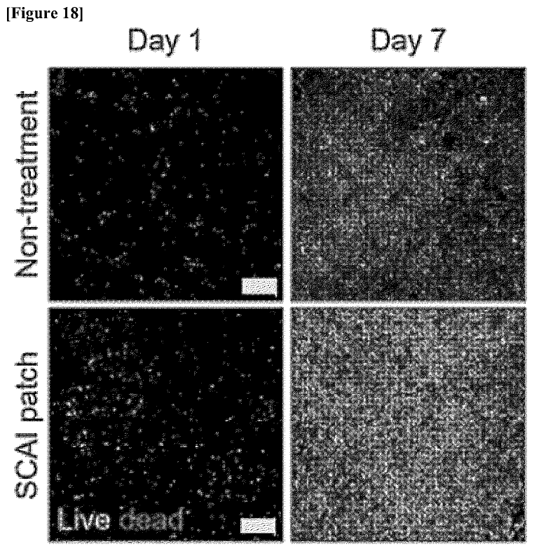

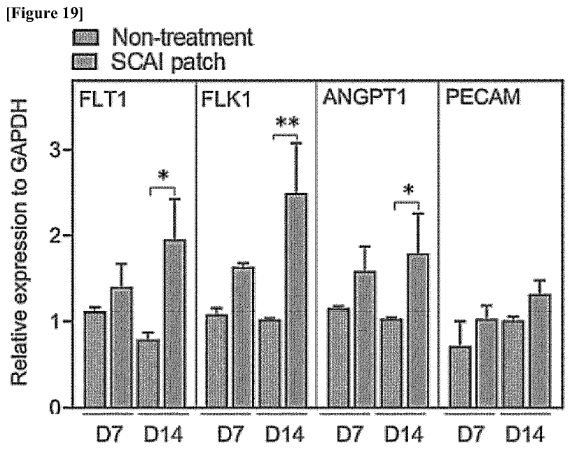

To evaluate the therapeutic potential of the SCAI patch by promoting angiogenesis, the biocompatibility and in vivo functions were verified through in vitro experiments. FIG. 17 shows an in vitro experimental result when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs). In the in vitro experiment of FIG. 17, HBMEC cells were cultured with an SCAI patch positioned on a transwell. As shown in FIG. 17, when the SCAI patch was applied to HBMECs and cultured for 7 days, no harmful effects were observed on cell viability, and it was confirmed that the proliferation rate of HBMECs was promoted compared to an untreated group. FIG. 18 shows a result of analyzing cell viability using a live/dead assay kit when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs) (scale bars: 50 μm). Through the live/dead staining analysis results in FIG. 18, the cell proliferation results in FIG. 17 were cross-validated. FIG. 19 shows a result of analyzing expression levels of genes related to angiogenesis when culturing an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B, which is applied with human brain microvascular endothelial cells (HBMECs). As shown in FIG. 19, in the early stage of angiogenesis, angiogenesis-related genes were expressed more in the group applied with the SCAI patch compared to the untreated group, but there was no significant difference. Meanwhile, the expression of all angiogenesis-related gene markers, including FLT1, FLK1, ANGPT1, and PECAM, increased rapidly 14 days after application of the SCAI patch. These results mean that the SCAI patch has a strong effect on promoting angiogenesis. VEGF and HGF released from the SCAI patch affect HBMEC cells, and at this time, the delayed release of HGF has a synergistic effect with VEGF to effectively generate vessels.

(4) In Vivo Experiment for Evaluating Cerebral Angiogenesis Behavior after SCAI Patch Transplantation

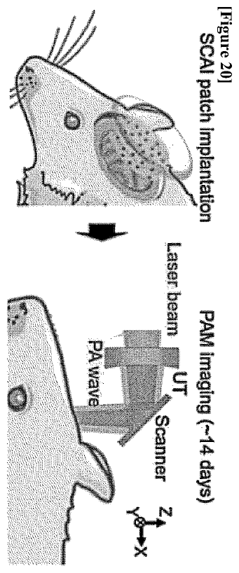

In order to evaluate the in vivo therapeutic potential of an SCAI patch consisting of an outer layer formed using HAVEM ink (4V0.5H) containing VEGF at a content of 0.05 μg/μl as ink A and an inner core layer formed using HAVEM ink (4V1H) containing HGF at a content of 0.05 μg/μl as ink B manufactured in the present study, the angiogenesis behavior of cerebral vessels induced by the SCAI patch was visualized over time using the OR-PAM system. FIG. 20 schematically illustrates an in vivo experiment consisting of a process of transplanting an SCAI patch spatiotemporally compartmentalized with VEGF and HGF manufactured in the present study into the cerebral cortex of SD rats and then monitoring a transplanted area using an OR-PAM system. The present inventors anesthetized rats, performed a craniotomy, removed the skull, and transplanted an SCAI patch into the cerebral region. Thereafter, a thin PDMS membrane was covered over the transplantation site to allow penetration of laser and photoacoustic (PA) ultrasound for monitoring. Thereafter, photoacoustic imaging was performed on the region of interest in the rat for 14 days to monitor blood circulation and cortical microvascular structural changes in the rat brain.

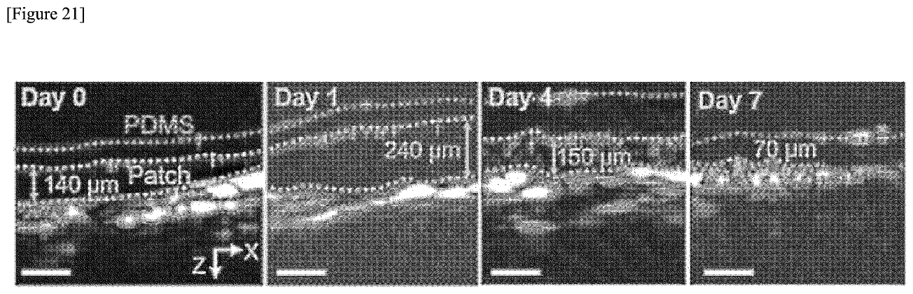

FIG. 21 shows images of cross-sections of an area where a patch is transplanted photographed using a photoacoustic (PA) B-mode in order to track changes in the SCAI patch over time in an in vivo experiment for evaluating a cerebral angiogenesis behavior after SCAI patch transplantation conducted in the present study (scale bars: 200 μm). In FIG. 21, since the PA signal is weak inside the semitransparent SCAI patch due to low absorption of a laser beam, a boundary of the patch was determined based thereon and highlighted with a yellow dotted line. As shown in FIG. 21, the patch thickness on day 0 of transplantation was indicated as 140 μm, similar to the thickness of the vitrified SCAI patch. One day after transplantation, the patch swelled with a cerebrospinal fluid to increase the patch thickness to 240 μm. Thereafter, as the patch was degraded, the thickness decreased to approximately 70 μm on day 7 of transplantation and the patch became virtually indistinguishable in a PA B-mode image. In addition, 14 days after transplantation, the patch was completely degraded and no patch was observed in a PA B-mode image (result image not shown).

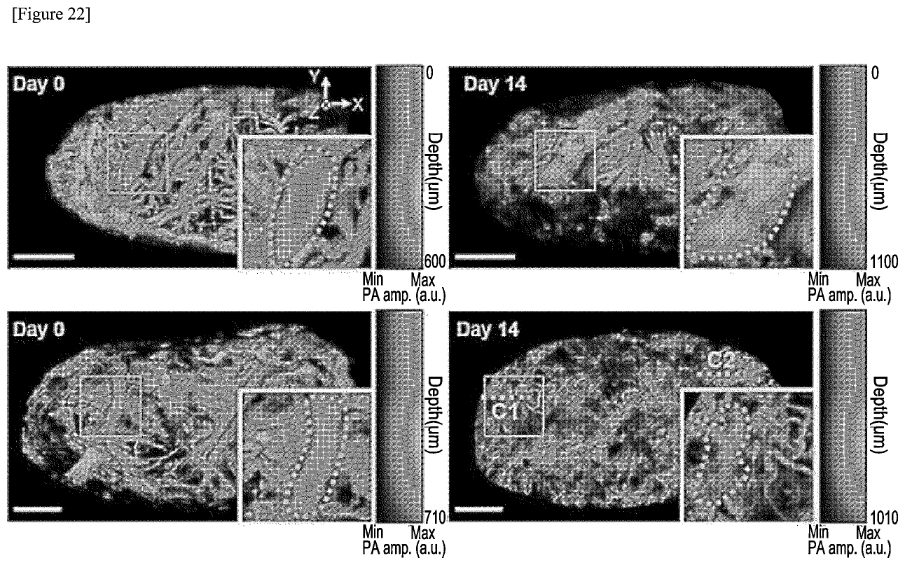

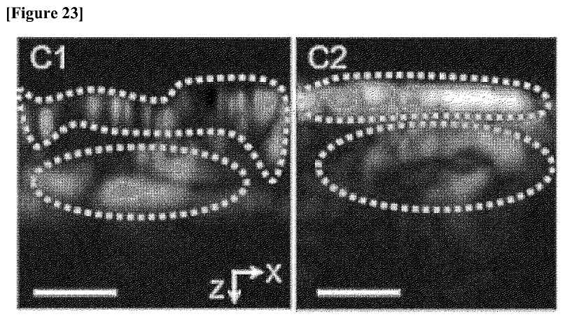

FIG. 22 shows changes in brain vascular structure in SD rats using photoacoustic (PA) maximum amplitude projection (MAP) images according to whether or not to be treated with SCAI patches in an in vivo experiment to evaluate cerebral angiogenesis behavior after SCAI patch transplantation performed in the present study. FIG. 23 is a cross-sectional PA B-mode image for areas C1 and C2 indicated by dotted lines in the image of a rat applied with the SCAI patch in FIG. 22 (scale bars: 400 μm). In the enlarged image of FIG. 23 (scale bars: 1 mm), large vessels are highlighted by elliptical curves formed by dots below. As shown in FIGS. 22 and 23, the transplanted SCAI patch induced angiogenesis, and changes in the brain microvascular structure were observed over time. After 14 days of transplantation, it could be seen that many microvascular structures were newly extended at the SCAI patch transplanted site, and many newly formed vessels with a diameter of 50 μm or less were observed due to a synergistic effect of the released VEGF and HGF. In contrast, no significant changes in microvascular structure were observed in the SCAI patch untreated group. In FIG. 22, the original vessels partially covered by the newly generated vessels may be seen in the enlarged image. The hidden original vessels were highlighted by dotted lines in areas C1 and C2 in the MAP image of FIG. 22, and were clearly seen in FIG. 23, which were PA B-mode images of the areas C1 and C2 shown in FIG. 22. In FIG. 23, the outlines of the original and newly formed vessels were indicated by an elliptical curve formed by points below and a curve formed by points above, respectively.

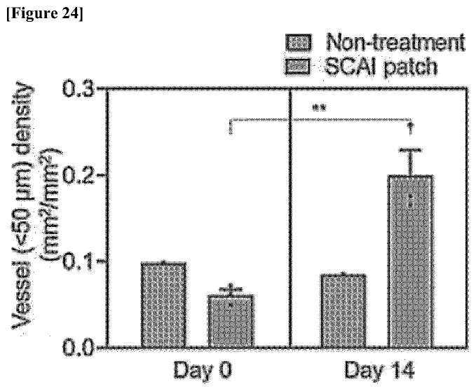

Quantitative analysis of the density of newly formed vessels was also performed. FIG. 24 shows results of quantitative analysis of the vascular density of SD rats according to whether or not to be treated with SCAI patches using optical resolution photoacoustic microscopy (OR-PAM) images in an in vivo experiment to evaluate a cerebral angiogenesis behavior after SCAI patch transplantation performed in the present study. The vascular density was determined as a ratio of the area of vessels with a diameter of less than 50 μm to the transplanted surface area. As shown in FIG. 24, when comparing the density obtained before and 14 days after SCAI patch transplantation, the vascular density after patch transplantation increased approximately 3.3 times from 0.0609±0.0052 mm²/mm² to 0.1999±0.0239 mm²/mm² on day 14 after transplantation, and these results suggest the therapeutic efficacy of the SCAI patch manufactured in the present study in promoting cerebral angiogenesis.

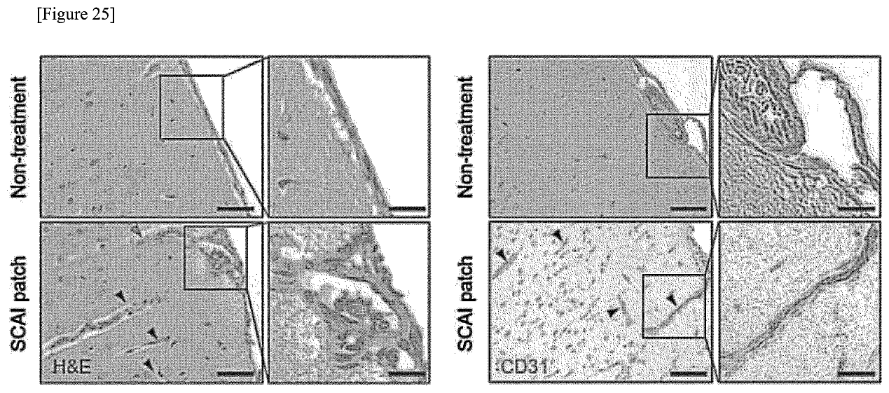

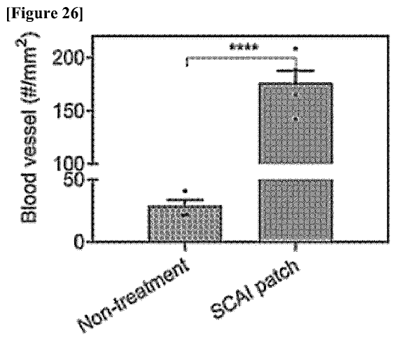

The present inventors also performed histological evaluation to verify the angiogenesis efficacy of the SCAI patch, which showed similar results to the content confirmed by PAM imaging. FIG. 25 shows histological analysis results for a patch-untreated group and an SCAI patch-transplanted group after 14 days of SCAI patch transplantation in H&E staining images and immunohistochemical (IHC) images (CD 31) in an in vivo experiment to evaluate a cerebral angiogenesis behavior after SCAI patch transplantation performed in the present study (scale bars: 50 μm). The scale bars of the enlarged image in FIG. 25 were 20 μm. As shown in FIG. 25, in the group transplanted with the SCAI patch, large vessels remained to supply blood to the surrounding tissues of the patch transplantation site after 14 days of patch transplantation, and numerous capillaries of various diameters and lengths were also observed (indicated by black arrows). Some of the vessels appeared to branch out from large original vessels, which was clear evidence of angiogenesis (indicated by gray arrows). Meanwhile, in the SCAI patch untreated group, no pattern of vessels extending from large vessels could be observed. In addition, the present inventors cross-validated the angiogenesis effect of the transplanted patch using immunohistochemistry (IHC) for a rat-specific vascular antibody and observed a similar trend to the H&E staining results (indicated by black arrows). In addition, the present inventors quantified the degree of angiogenesis by counting the number of vessels per unit area (1 mm²). FIG. 26 shows results of quantitative analysis of the number of vessels per unit area in a patch-untreated group and an SCAI patch-transplanted group using immunohistochemical (IHC) images after 14 days of SCAI patch transplantation in an in vivo experiment to evaluate a cerebral angiogenesis behavior after SCAI patch transplantation performed in the present study. As shown in FIG. 26, the number of vessels in the SCAI patch transplanted group was 167±13, and the number of vessels in the untreated group was 29±5, and as a result, in the SCAI patch transplanted group, approximately 5.76 times more vessels per 1 mm² were newly generated. In summary, it can be concluded that the SCAI patch promotes vascularization and angiogenesis and maintains the therapeutic efficacy in vivo for at least 14 days.

### 3. DISCUSSION AND CONCLUSION

The present inventors have manufactured a flexible patch-type drug delivery system capable of releasing two angiogenic factors according to an angiogenesis stage to promote cerebral angiogenesis in the brain region. The main features of the manufactured SCAI patch are as follows:

(1) The HAVEM ink used to form the patch may variously control release patterns by the bonding density between a methacrylate functional group and an amine functional group.

(2) Angiogenic growth factors may be spatiotemporally compartmentalized and sequentially sustained-released using 3D printing technology.

Conventional bioprocessing strategies (e.g., mold casting, gas foaming, and freeze-drying) have been widely applied to produce a biomaterial-based drug delivery system. However, the method inevitably involves several complex processes. In order to improve the problem, the present inventors applied 3D printing technology to manufacture a patch with a structure in which the inner and outer layers were spatially separated only in a single process without going through multiple physical processes. When reducing the number of process steps, the overall manufacturing time is reduced to help in maintaining higher biological activity of the growth factors. In addition, traditional approaches generally often perform treating toxic solutions or additional processes under harsh environmental factors (e.g., pH, temperature). The processes also reduce the stability of protein-based drugs. However, since the HAVEM ink manufactured by the present inventors does not form other chemically active substances including radicals as intermediate substances during the synthesis process and has mild crosslinking conditions without requiring a catalyst, the HAVEM ink may also contribute to maintaining the bioactivity of the growth factors.

Patches with spatiotemporally compartmentalized dual angiogenic factors have a significant impact on inducing cerebral angiogenesis. The present inventors investigated the in vivo therapeutic efficacy using the OR-PAM system to monitor time-dependent changes in the microvasculature of the rat cerebral cortex for 14 days. This imaging method is a vascular imaging method without requiring the use of labeling substances because the method obtains vascular images by measuring PA waves generated by red blood cells present in the vascular system. The OR-PAM system may identify functional, integrated, newly formed vessels in a more effective and noninvasive manner than conventional vascular imaging techniques.

The SCAI patch manufactured by the present inventors may serve as a reliable guide for further investigation of the possibility of neovascularization in a cerebral ischemic region. In addition, an angiogenic growth factor sustained-release patch system manufactured by the present inventors can also be applied to treatments targeting intractable chronic cerebral ischemic diseases such as Moyamoya disease. Moyamoya disease is a progressive occlusive arteriopathy characterized by occlusion of the internal carotid artery and its branches. Treatment of patients with Moyamoya disease is mainly performed through surgical operations (direct anastomosis and indirect anastomosis). However, pediatric patients cannot undergo direct anastomosis because the superficial temporal artery (STA) is too thin to be directly anastomosed to the middle cerebral artery, and indirect revascularization strategies, such as encephaloduroarteriosynangiosis (EDAS), are widely accepted as treatment strategies for pediatric Moyamoya disease. The effect of this surgical technique is generally known to vary depending on the level of growth factors and angiogenesis ability of the cerebrospinal fluid. The SCAI patch manufactured by the present inventors may exhibit a beneficial effect of promoting angiogenesis between the superficial temporal artery (STA) and the dural blood vessels after indirect surgery even only by placing the patch near the site.

The study results highlight the outstanding potential of a spatiotemporally compartmentalized cerebral angiogenesis-inducing patch manufactured using HAVEM ink. The HAVEM ink is a 3D printing ink with controlled crosslinking density through an aza-Michael addition reaction. In the present study, a decellularized extracellular matrix (dECM)-based hybrid ink with controlled crosslinking density was synthesized through the aza-Michael addition reaction, and a complex patch-shaped structure was manufactured using 3D printing technology based on the improved printability of the hybrid ink. Hybrid inks with higher crosslinking density have smaller pore sizes due to tighter bonding, and may effectively reduce the release rate of growth factors. In addition, when using the 3D printing technology, it is possible to manufacture a physically compartmentalized structure capable of controlling the release of growth factors loaded within the hybrid ink. Therefore, the SCAI patch manufactured in the present study may effectively induce angiogenesis necessary for the treatment of ischemic diseases by continuously sustained-releasing two types of angiogenic growth factors. The SCAI patch-type drug delivery platform developed in the present study overcome the problems occurring in conventional long-term drug delivery approaches. Patches formed from HAVEM ink containing a vessel-derived decellularized extracellular matrix (VdECM) and methacrylated hyaluronic acid (HAMA) are made of the actual corresponding tissue-derived materials to have soft and flexible characteristics and have versatility in manufacturing. The SCAI patch manufactured in the present study may be used as a promising alternative therapeutic agent because the SCAI patch may effectively induce angiogenesis when applied to patients with various acute or chronic cerebral ischemic diseases. In addition, when applying various different types of drugs to the spatiotemporally compartmentalized patch or further forming additional layers capable of loading additional drugs, a spatiotemporally compartmentalized patch can effectively function as a drug delivery platform that needs to deliver drugs locally by controlling a dose and can be used for various applications such as treatment of ischemic heart disease, diabetes, etc., and coronavirus vaccines.

As described above, the present disclosure has been described through Examples above, but the present disclosure is not necessarily limited thereto, and various modifications can be made without departing from the scope and spirit of the present disclosure. Therefore, the scope of the present disclosure should be construed to include all embodiments falling within the scope of claims appended hereto.

1. An extracellular matrix-based hybrid ink for 3D printing, the hybrid ink comprising an extracellular matrix having an amine group and a modified hyaluronic acid introduced with an ethylenically unsaturated bond functional group,
wherein the extracellular matrix and the modified hyaluronic acid are present in a crosslinked state by a chemical bond formed between at least some of the amine groups present in the extracellular matrix and the ethylenically unsaturated bond functional group present in the modified hyaluronic acid.

2. The extracellular matrix-based hybrid ink for 3D printing of claim 1, wherein the extracellular matrix is a vessel-derived decellularized extracellular matrix.

3. The extracellular matrix-based hybrid ink for 3D printing of claim 1, wherein the ethylenically unsaturated bond functional group is selected from a vinyl group, an acrylic group, or a methacrylic group.

4. The extracellular matrix-based hybrid ink for 3D printing of claim 1, wherein the modified hyaluronic acid is selected from methacrylated hyaluronic acid or acrylated hyaluronic acid.

5. The extracellular matrix-based hybrid ink for 3D printing of claim 1, wherein the chemical bond between the amine group present in the extracellular matrix and the ethylenically unsaturated bond functional group present in the modified hyaluronic acid is formed by an aza-Michael addition reaction.

6. The extracellular matrix-based hybrid ink for 3D printing of claim 1, wherein a weight ratio of the extracellular matrix to the modified hyaluronic acid is 1:0.01 to 1:0.8.

7. The extracellular matrix-based hybrid ink for 3D printing of claim 1, wherein a concentration of the extracellular matrix in the hybrid ink is 1 to 10% (w/v), and a concentration of the modified hyaluronic acid is 0.01 to 8% (w/v).

8. The extracellular matrix-based hybrid ink for 3D printing of claim 1, further comprising:
a growth factor.

9. The extracellular matrix-based hybrid ink for 3D printing of claim 8, wherein the growth factor consists of at least one selected from the group consisting of vascular endothelial growth factor (VEGF), hepatocyte growth factor (HGF), epithermal growth factor (EGF), insulin-like growth factor (IGF), erythropoietin (EPO), fibroblast growth factor (FGF), brain-derived neurotrophic factor (BDNF), nerve growth factor (NGF), keratinocyte growth factor (KGF), interleukin (IL), colony-stimulating factor (CSF), angiopoietin (ANG), platelet-derived growth factor (PDGF), placental growth factor (PGF), transforming growth factor-α (TGF-α), transforming growth factor-β (TGF-β), matrix metalloproteinase (MMP), and bone morphogenetic protein (BMP).

10. The extracellular matrix-based hybrid ink for 3D printing of claim 8, wherein a concentration of the growth factor in the hybrid ink is 0.001 to 1 g/l.

11. A method for manufacturing an extracellular matrix-based hybrid ink for 3D printing, the method comprising:
preparing an extracellular matrix solution having a pH of 6.5 to 7.5; and
adding and uniformly mixing a modified hyaluronic acid introduced with an ethylenically unsaturated bond functional group to the extracellular matrix solution, and then performing an aza-Michael addition reaction to obtain an ink in which the extracellular matrix and the modified hyaluronic acid are in a crosslinked state.

12. The method of claim 11, wherein the extracellular matrix is a vessel-derived decellularized extracellular matrix.

13. The method of claim 11, wherein the ethylenically unsaturated bond functional group is selected from a vinyl group, an acrylic group, or a methacrylic group.

14. The method of claim 11, wherein the modified hyaluronic acid is selected from methacrylated hyaluronic acid or acrylated hyaluronic acid.

15. The method of claim 11, wherein a chemical bond is formed between at least some of amine groups present in the extracellular matrix and the ethylenically unsaturated bond functional group present in the modified hyaluronic acid by the aza-Michael addition reaction.

16. The method of claim 11, wherein the modified hyaluronic acid is added to the extracellular matrix solution in an amount of 1 to 80 parts by weight based on 100 parts by weight of the extracellular matrix.

17. The method of claim 11, wherein a concentration of the extracellular matrix in the extracellular matrix solution is 1 to 10% (w/v), and the modified hyaluronic acid is added to the extracellular matrix solution to be a concentration of 0.01 to 8% (w/v).

18. The method of claim 11, wherein the aza-Michael addition reaction is performed at a temperature condition of 1 to 10° C. for 2 to 24 hours.

19. The method of claim 11, further comprising:
adding and mixing a growth factor to the ink.

20. The method of claim 19, wherein the growth factor is added to the ink at a concentration of 0.001 to 1 g/l.
